(19) United States
(12) Patent Application Publication
(10) Pub. No.: US 2006/0257484 A1
(43) Pub. Date: Nov. 16, 2006

(54) Combination of tramadol and substances that comprise gabapentin

(76) Inventors: Stephen S. Hwang, Palo Alto, CA (US); Sandra Chaplan, San Diego, CA (US); Dong Yan, Cupertino, CA (US); Patrick S.L. Wong, Burlingame, CA (US); David Abraham, Menlo Park, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/404,293

(22) Filed: Apr. 13, 2006

Related U.S. Application Data
(60) Provisional application No. 60/673,036, filed on Apr. 19, 2005.

(51) Int. Cl. A61K 31/195; A61K 31/137; A61K 9/24

(52) U.S. Cl. 424/473; 514/561; 514/650

(57) ABSTRACT

Disclosed are substances, compositions, dosage forms and methods that comprise tramadol and substances that comprise gabapentin.

### CROSS REFERENCE TO RELATED APPLICATIONS

The present invention claims the benefit under 35 USC 119(e) to U.S. Provisional patent application 60/673,036 filed Apr. 19, 2005.

### FIELD OF THE INVENTION

The invention relates to substances, compositions, dosage forms and methods that comprise tramadol and substances that comprise gabapentin.

### BACKGROUND

Scientific understanding about the pathogenesis of neuropathic pain has grown over the last decades as basic research with animal models of neuropathic pain and human clinical trials have revealed the pathophysiological and biochemical changes in the nervous system due to an insult or disease (Backonja, M. M., Clin. J. Pain, 16(2):S67-72 (2000)). Neuropathic pain is a chronic pain, often experienced by cancer patients, stroke victims, elderly persons, diabetics (as painful diabetic neuropathy), persons with herpes zoster (shingles), as postherpetic neuralgia, and in persons with neurodegenerative diseases, such as amyotrophic lateral sclerosis (ALS). Clinical characteristics of neuropathic pain include burning, spontaneous pain, shooting pain, and evoked pains. Distinct pathophysiological mechanisms lead to specific sensory symptoms, such as dynamic mechanical allodynia and cold hyperalgesia.

Therapies for treatment of neuropathic pain include use of traditional analgesics such as nonsteroidal anti-inflammatory drugs, and opioids, as well as other agents including anticonvulsants and tricyclic antidepressants (Max, M. B., Ann. Neurol., 35 (Suppl):S50-S53 (1994); Raja, S. N. et al., Neurology, 59:1015 (2002); Galer, B. S. et al., Pain, 80:533 (1999)). Many patients are refractory to these and other treatments because of inadequate pain relief or intolerable side effects. In other words, current neuropathic pain treatments have a poor therapeutic index.

Furthermore, the nature of pharmacologic intervention in neuropathic pain is sub-optimal in the frequency of dosing. A variety of medicines for treatment of neuropathic pain such as gabapentin and tramadol must be dosed to a patient several times per day. This is especially undesirable for pain relief, because dosing regimens are inconvenient for patients and the pain may lose control when the plasma drug concentration drops below the therapeutic level. The inconvenience can lead to low compliance and poor pain control. The loss of pain control could lead patients to perceive a potentially successful treatment as a failure. A desirable dosing frequency for neuropathic pain is once per day (qd).

Accordingly, neuropathic pain treatment substances, compositions, dosage forms, and methods with improved therapeutic index, and qd dosing are needed.

### SUMMARY OF THE INVENTION

In an embodiment, the invention relates to an oral dosage form comprising: an oral controlled delivery dosing structure comprising structure that controllably delivers tramadol and a substance that comprises gabapentin; and wherein the weight ratio of gabapentin equivalent to tramadol equivalent present in the oral dosage form ranges from about 0.75:1 to about 6.5:1; and wherein the total weight of the tramadol and substance that comprises gabapentin present in the oral dosage form is less than about 1500 milligrams; and wherein the oral controlled delivery dosing structure is adapted to controllably deliver the substance that comprises gabapentin at a rate that is effective to, after a single administration of the oral dosage form to a patient, maintain a gabapentin plasma drug concentration that is at least about twenty-five percent of a gabapentin Cmax throughout a window of at least about fifteen hours duration after a time at which the gabapentin Cmax occurs.

In an embodiment, the invention relates to an oral dosage form comprising: an oral controlled delivery dosing structure comprising structure that controllably delivers tramadol and a substance that comprises gabapentin; wherein the weight ratio of gabapentin equivalent to tramadol equivalent present in the oral dosage form ranges from about 0.75:1 to about 6.5:1; and wherein the total weight of the tramadol and substance that comprises gabapentin present in the oral dosage form is less than about 1500 milligrams; and wherein the controlled delivery dosing structure is adapted to controllably deliver the substance that comprises gabapentin contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs, wherein the wt % is based on the total weight of the substance that comprises gabapentin present in the controlled delivery dosage form.

In another embodiment, the invention relates to an oral dosage form comprising: an oral controlled delivery dosing structure comprising structure that controllably delivers tramadol and a substance that comprises gabapentin; wherein the weight ratio of gabapentin equivalent to tramadol equivalent present in the oral dosage form ranges from about 0.75:1 to about 6.5:1; and wherein the total weight of the tramadol and substance that comprises gabapentin present in the oral dosage formn is less than about 1500 milligrams; and wherein the controlled delivery dosing structure is adapted to controllably deliver the portion of the substance that comprises tramadol contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs, wherein the wt % is based on the total weight of the tramadol present in the controlled delivery dosage form.

In still another embodiment, the invention relates to an oral controlled delivery dosage form comprising: an oral controlled delivery dosing structure comprising structure that controllably delivers (i) a substance that comprises gabapentin, and (ii) tramadol; wherein the oral controlled delivery dosing structure is adapted to controllably deliver the substance that comprises gabapentin at a release rate that satisfies the following relationship: Rate₀₋₃=(1/F) * Rate₃₋₁₀ wherein Rate₀₋₃ represents a mean release rate for about a three hour period immediately following administration of the dosage form, Rate₃₋₁₀ represents a mean release rate for a period from about three hours immediately following administration of the oral dosage form to about ten hours immediately following administration of the oral dosage form, and F=X/Y, wherein X=a colonic bioavailability of gabapentin and Y=upper gastrointestinal tract bioavailability of gabapentin.

In an embodiment, the invention relates to a method comprising (1) providing an oral dosage form comprising: an oral controlled delivery dosing structure comprising structure that controllably delivers tramadol and a substance that comprises gabapentin; and wherein the weight ratio of gabapentin equivalent to tramadol equivalent present in the oral dosage form ranges from about 0.75:1 to about 6.5:1; and wherein the total weight of the tramadol and substance that comprises gabapentin present in the oral dosage form is less than about 1500 milligrams; and wherein the oral controlled delivery dosing structure is adapted to controllably deliver the substance that comprises gabapentin at a rate that is effective to, after a single administration of the oral dosage form to a patient, maintain a gabapentin plasma drug concentration that is at least about twenty-five percent of a gabapentin Cmax throughout a window of at least about fifteen hours duration after a time at which the gabapentin Cmax occurs; and (2) administering the oral dosage form to a patient.

In yet another embodiment, the invention relates to a method comprising: (1) providing an oral dosage form comprising: an oral controlled delivery dosing structure comprising structure that controllably delivers tramadol and a substance that comprises gabapentin; wherein the weight ratio of gabapentin equivalent to tramadol equivalent present in the oral dosage form ranges from about 0.75:1 to about 6.5:1; and wherein the total weight of the tramadol and substance that comprises gabapentin present in the oral dosage form is less than about 1500 milligrams; and wherein the controlled delivery dosing structure is adapted to controllably deliver the substance that comprises gabapentin contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs, wherein the wt % is based on the total weight of the substance that comprises gabapentin present in the controlled delivery dosage form; and (2) administering the oral dosage form to a patient.

In an embodiment, the invention relates to a method comprising: (1) providing an oral dosage form comprising: an oral controlled delivery dosing structure comprising structure that controllably delivers tramadol and a substance that comprises gabapentin; wherein the weight ratio of gabapentin equivalent to tramadol equivalent present in the oral dosage form ranges from about 0.75:1 to about 6.5:1; and wherein the total weight of the tramadol and substance that comprises gabapentin present in the oral dosage form is less than about 1500 milligrams; and wherein the controlled delivery dosing structure is adapted to controllably deliver the portion of the substance that comprises tramadol contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs, wherein the wt % is based on the total weight of the tramadol present in the controlled delivery dosage form; and (2) administering the oral dosage form to a patient.

In a further embodiment, the invention relates to a method comprising: (1) providing an oral controlled delivery dosage form comprising an oral controlled delivery dosing structure comprising structure that controllably delivers: (i) a substance that comprises gabapentin, and (ii) tramadol; wherein the oral controlled delivery dosing structure is adapted to controllably deliver the substance that comprises gabapentin at a release rate that satisfies the following relationship: Rate₀₋₃=(1/F) * Rate₃₋₁₀ wherein Rate₀₋₃ represents a mean release rate for about a three hour period immediately following administration of the dosage form, Rate₃o₁₀ represents a mean release rate for a period from about three hours immediately following administration of the oral dosage form to about ten hours immediately following administration of the oral dosage form, and F=X/Y, wherein X=a colonic bioavailability of gabapentin and Y=upper gastrointestinal tract bioavailability of gabapentin; and (2) administering the dosage form to a patient.

In still a further embodiment, the invention relates to a pharmaceutical composition comprising a substance comprising a complex that comprises (i) gabapentin and (ii) a transport moiety; and tramadol.

In yet another embodiment, the invention relates to a method comprising: (1) providing a pharmaceutical composition comprising a substance comprising a complex that comprises (i) gabapentin and (ii) a transport moiety; and tramadol; and (2) administering the pharmaceutical composition to a patient.

### BRIEF DESCRIPTION OF THE FIGURES

The following figures are not drawn to scale, and are set forth to illustrate various embodiments of the invention.

FIG. 1 shows a diagram of a liquid osmotic dosage form.

FIG. 2 shows a diagram of a liquid osmotic dosage form.

FIG. 3 shows a diagram of an osmotic dosage form.

FIG. 4 shows a diagram of an elementary osmotic pump dosage form.

FIGS. 5A-5C show diagrams of a controlled release dosage form.

### DETAILED DESCRIPTION

### I. DEFINITIONS

All documents cited to herein are incorporated by reference in their entirety for all purposes, as if reproduced fully herein.

The present invention is best understood by reference to the following definitions, the drawings and exemplary disclosure provided herein.

By “ascending rate of release” is meant a rate of release wherein the amount of drug released as a function of time increases over a period of time, preferably continuously and gradually. Preferably, the rate of drug released as a function of time increases in a steady (rather than step-wise) manner. More preferably, an ascending rate of release may be characterized as follows. The rate of release as a function of time for a dosage form is measured and plotted as % drug release versus time or as milligrams of drug released/hour versus time. An ascending rate of release is characterized by an average rate (expressed in mg of drug per hour) wherein the rate within a given two hour span is higher as compared with the previous two hour time span, over the period of time of about 2 hours to about 12 hours, preferably, about 2 hours to about 18 hours, more preferably about 4 hours to about 12 hours, more preferably still, about 4 hours to about 18 hours. Preferably, the increase in average rate is gradual such that less than about 30% of the dose is delivered during any 2 hour interval, more preferably, less than about 25% of the dose is delivered during any 2 hour interval. Preferably, the ascending release rate is maintained until at least about 50%, more preferably until at least about 75% of the drug in the dosage form has been released.

By “area under the curve” or “AUC” is meant the area as measured under a plasma drug concentration curve. Often, the AUC is specified in terms of the time interval across which the plasma drug concentration curve is being integrated, for instance AUC_{start-finish}. Thus, AUC₀₋₄₈ refers to the AUC obtained from integrating the plasma concentration curve over a period of zero to 48 hours, where zero is conventionally the time of administration of the drug or dosage form comprising the drug to a patient. AUCₜ refers to area under the plasma concentration curve from hour 0 to the last detectable concentration at time t, calculated by the trapezoidal rule. AUC_{inf} refers to the AUC value extrapolated to infinity, calculated as the sum of AUCₜ and the area extrapolated to infinity, calculated by the concentration at time t (Ct) divided by k. (If the t_{1/2} value was not estimable for a subject, the mean t_{1/2} value of that treatment may be used to calculate AUC_{inf}.). “k” is defined as the apparent elimination rate constant is estimated by linear regression of the log-transformed plasma concentration during the terminal log-linear decline phase

By “C” is meant the concentration of a drug in blood plasma, or serum, of a subject, generally expressed as mass per unit volume, typically nanograms per milliliter. For convenience, this concentration may be referred to herein as “drug plasma concentration”, “plasma drug concentration” or “plasma concentration” which is intended to be inclusive of drug concentration measured in any appropriate body fluid or tissue. The plasma drug concentration at any time following drug administration is referenced as Ctime, as in C9h or C24h, etc.

By “Cmax” is meant the mean maximum drug plasma concentration following administration of a single dose of the drug to patients.

By “colonic bioavailability of gabapentin” is meant the AUC_{inf} obtained when a dose of a substance comprising gabapentin is administered to the colon divided by the AUC_{inf} obtained when a dose of a substance comprising gabapentin is administered intravenously.

By “composition” is meant a drug in combination with additional active pharmaceutical ingredients, and optionally in combination with inactive ingredients, such as pharmaceutically-acceptable carriers, excipients, suspension agents, surfactants, disintegrants, binders, diluents, lubricants, stabilizers, antioxidants, osmotic agents, colorants, plasticizers, and the like.

By “complex” is meant a substance comprising a drug moiety and a transport moiety associated by a tight-ion pair bond. A drug-moiety-transport moiety complex can be distinguished from a loose ion pair of the drug moiety and the transport moiety by a difference in octanol/water partitioning behavior, characterized by the following relationship:

ΔLogD=Log D (complex)—Log D (loose-ion pair)≧0.15 (Equation 1) wherein: D, the distribution coefficient (apparent partition coefficient), is the ratio of the equilibrium concentrations of all species of the drug moiety and the transport moiety in octanol to the same species in water (deionized water) at a set pH (typically about pH=5.0 to about pH=7.0) at 25 degrees Celsius. Log D (complex) is determined for a complex of the drug moiety and transport moiety prepared according to the teachings herein. Log D (loose-ion pair) is determined for a physical mixture of the drug moiety and the transport moiety in deionized water. Log D can be determined experimentally or may be predicted for loose-ion pairs using commercially available software packages (e.g., ChemSilico, Inc., Advanced Chemistry Development Inc).

For instance, the octanol/water apparent partition coefficient (D=C_{octanol}/C_{water}) of a putative complex (in deionized water at 25 degree Celsius) can be determined and compared to a 1:1 (mol/mol) physical mixture of the transport moiety and the drug moiety in deionized water at 25 degree Celsius. If the difference between the Log D for the putative complex (D+T−) and the Log D for the 1:1 (mol/mol) physical mixture, D⁺∥T⁻ is determined is greater than or equal to 0.15, the putative complex is confirmed as being a complex according to the invention.

In preferable embodiments, A Log D≧0.20, and more preferably A Log D≧0.25, more preferably still A Log D≧0.35.

By “controlled delivery ” or “controllable delivery” is meant continuous or discontinuous release of a drug, wherein the drug is released at (a) a controlled rate over (b) a prolonged period of time and in (c) a manner that provides for improved drug absorption as compared to the absorption of the drug in an immediate release dosage form.

Controlled delivery technologies comprise technologies that (1) provide improved upper G.I. tract and/or lower G.I. tract absorption of gabapentin, (2) provide upper G.I. tract and/or lower G.I. tract delivery of gabapentin (including various improved absorption forms of gabapentin), and (3) provide upper G.I. tract and/or lower G.I. tract delivery of tramadol. In a preferred embodiment, controlled delivery technologies comprise technologies that improve the lower G.I. tract absorption of gabapentin. Technologies that improve the upper G.I. tract and/or lower G.I. tract absorption of gabapentin include, but are not limited to, (i) complexation of forms of gabapentin with transport moieties and/or delivery of such complexes to the upper and lower G.I. tract, preferably the lower G.I. tract; and (ii) forming prodrugs of forms of gabapentin with improved upper and lower G.I. tract, preferably lower G.I. tract, absorption and/or delivery of such prodrugs to the upper and lower G.I. tract, preferably the lower G.I. tract. In a preferred embodiment, tramadol and/or gabapentin are controllably delivered by complexation of gabapentin with alkyl sulfate salts coupled with delivery of tramadol and such complexes to the upper and lower G.I. tract.

By “dosage form” is meant a pharmaceutical composition inca medium, carrier, vehicle, or device suitable for administration to a patient.

By “dosing structure” is meant a structure suitable for pharmaceutical dosing to a patient.

By “drug” or “drug moiety” is meant a drug, compound, or agent, or a residue of such a drug, compound, or agent that provides some pharmacological effect when administered to a subject. For use in forming a complex, the drug comprises a(n) acidic, basic, or zwitterionic structural element, or a(n) acidic, basic, or zwitterionic residual structural element. In embodiments according to the invention, drug moieties that comprise acidic structural elements or acidic residual structural elements are complexed with transport moieties that comprise basic structural elements or basic residual structural elements. In embodiments according to the invention, drug moieties that comprise basic structural elements or basic residual structural elements are complexed with transport moieties that comprise acidic structural elements or acidic residual structural elements. In embodiments according to the invention, drug moieties that comprise zwitterionic structural elements or zwitterionic residual structural elements are complexed with transport moieties that comprise either acidic or basic structural elements, or acidic or basic residual structural elements. In an embodiment, the pKa of an acidic structural element or acidic residual structural element is less than about 7.0, preferably less than about 6.0. In an embodiment, the pKa of a basic structural element or basic residual structural element is greater than about 7.0, preferably greater than about 8.0. Zwitterionic structural elements or zwitterionic residual structural elements are analyzed in terms of their individual basic structural element or basic residual structural element or their acidic structural element or acidic residual structural element, depending upon how the complex with the transport moiety is to be formed.

By “orifice” or “exit orifice” is meant means suitable for releasing the active agent from the dosage form. The expression includes aperture, hole, bore, pore, porous element, porous overlay, porous insert, hollow fiber, capillary tube, microporous insert, microporous overlay, and the like.

By “fatty acid” is meant any of the group of organic acids of the general formula CH₃(CₙHₓ)COOH where the hydrocarbon chain is either saturated (x=2n, e.g. palmitic acid, CH₃C₁₄H₂₈COOH) or unsaturated (for monounsaturated, x=2n-2, e.g. oleic acid, CH₃C₁₆H₃₀COOH).

By “gabapentin” is meant gabapentin, and pharmaceutically acceptable salts thereof By “substances that comprise gabapentin” is meant substances that include gabapentin as the primary pharmacological entity within the substance. Accordingly, such substances include, but are not limited to, complexes of gabapentin with alkyl sulfate salts; and prodrugs of gabapentin possessing improved lower G.I. absorption.

By “gabapentin equivalent” is meant that portion of the substance that comprises gabapentin that is actually gabapentin. As the molecular weight is different for various forms of substances that comprise gabapentin, it is confusing to report the dose for a dosage form according to the weight of the substance. It is preferred to report the dose as the gabapentin equivalent, i.e. the weight equivalent of gabapentin present in the substance. For instance, the molecular weight of gabapentin-lauryl sulfate is 437.64, while the molecular weight of gabapentin is 171.24. To dose 100 mg weight of gabapentin equivalent, one would need to dose 255.6 mg of gabapentin-lauryl sulfate. On this basis, certain embodiments according to the invention may comprise a gabapentin equivalent present in the dosage form ranging from about 50 mg to about 2000 mg, preferably from about 50 mg to about 900 mg, and more preferably from about 100 mg to about 600 mg. Particular dosage forms may contain about 40 mg, about 50 mg, about 100 mg, about 150 mg, about 200 mg, about 300 mg, about 400 mg, about 500 mg, about 750 mg, or about 1000 mg weight equivalents in a given dosage form.

By “immediate-release” is meant a dose of a drug that is substantially completely released from a dosage form within a time period of about 1 hour or less and, preferably, about 30 minutes or less. Certain controlled delivery dosage forms may require a short time period following administration in which to begin to release drug. In embodiments, wherein the slight delay in initial drug release is not desirable, an immediate-release overcoat can be applied to the surface of the controlled delivery dosage form. An immediate-release dose of drug applied as a coating on the surface of a dosage form refers to a dose of drug prepared in a suitable pharmaceutically acceptable carrier to form a coating solution that will dissolve rapidly upon administration thereby providing an immediate-release dose of drug. As is known in the art, such immediate release drug overcoats can contain the same or a different drug or drugs as is contained within the underlying dosage form.

By “intestine” or “gastrointestinal (G.I.) tract” is meant both the upper gastrointestinal tract and the lower gastrointestinal tract.

By “loose ion-pair” is meant a pair of ions that, at physiologic pH and in an aqueous environment, are readily interchangeable with other loosely paired or free ions that may be present in the environment of the loose ion pair. Loose ion-pairs can be found experimentally by noting interchange of a member of a loose ion-pair with another ion, at physiologic pH and in an aqueous environment, using isotopic labeling and NMR or mass spectroscopy. Loose ion-pairs also can be found experimentally by noting separation of the ion-pair, at physiologic pH and in an aqueous environment, using reverse phase HPLC. Loose ion-pairs may also be referred to as “physical mixtures,” and are formed by physically mixing the ion-pair together in a medium.

By “lower gastrointestinal tract”, “lower G.I. tract”, “large intestine”, “colon”, or “colonic” is meant the ascending colon, transverse colon, descending colon, sigmoid colon, and/or rectum.

By “patient” is meant an animal, preferably a mammal, more preferably a human, in need of therapeutic intervention.

By “pharmaceutically acceptable salt” is meant any salt of a low solubility and/or low dissolution rate pharmaceutical agent whose cation or anion does not contribute significantly to the toxicity or pharmacological activity of the salt, and, as such, they are the pharmacological equivalents of the low solubility and/or low dissolution rate free acid pharmaceutical agent.

By “pharmaceutical composition” is meant a composition suitable for administration to a patient in need thereof.

By “prolonged period of time” is meant a continuous period of time of greater than about 1 hour, preferably, greater than about 4 hours, more preferably, greater than about 8 hours, more preferably greater than about 10 hours, more preferably still, greater than about 14 hours, most preferably, greater than about 14 hours and up to about 24 hours.

By “rate of release” or “release rate” of a drug refers to the quantity of drug released from a dosage form per unit time, e.g., milligrams of drug released per hour (mg/hr). Drug release rates for dosage forms are typically measured as an in vitro rate of drug release, i.e., a quantity of drug released from the dosage form per unit time measured under appropriate conditions and in a suitable fluid. By “mean rate of release” is meant the mean release rate determined over a specified period. In a preferred embodiment, the period begins at some point following dosing, and continues during a relatively linear portion of the release of the drug(s) from the dosage form.

The release rates referred to herein are determined by placing a dosage form to be tested in de-ionized water in metal coil or metal cage sample holders attached to a USP Type VII bath indexer in a constant temperature water bath at 37° C. Aliquots of the release rate solutions, collected at pre-set intervals, are then injected into a chromatographic system fitted with an ultraviolet or refractive index detector to quantify the amounts of drug released during the testing intervals.

An alternative release rate test method may performed using the Distek 5100 (USP apparatus 2 paddle tester) in 900 mL artificial gastric fluid (AGF, pH=1.2). The temperature of the dissolution medium is maintained at 37° C. and the paddle speed is 100 rpm.

As used herein a drug release rate obtained at a specified time refers to the in vitro release rate obtained at the specified time following implementation of the release rate test. The time at which a specified percentage of the drug within a dosage form has been released from said dosage form is referred to as the “Tx” value, where “x” is the percent of drug that has been released. For example, a commonly used reference measurement for evaluating drug release from dosage forms is the time at which 70% of drug within the dosage form has been released. This measurement is referred to as the “T70” for the dosage form. Preferably, T70 is greater than or equal to about 8 hours, more preferably, T70 is greater than or equal to about 12 hours, more preferably still, T70 is greater than to equal to about 16 hours, most preferably, T70 is greater than or equal to about 20 hours. In one embodiment, T70 is greater than or equal to about 12 hours and less than about 24 hours. In another embodiment, T70 is greater than or equal to about 8 hours and less than about 16 hours.

By “residual structural element” is meant a structural element that is modified by interaction or reaction with another compound, chemical group, ion, atom, or the like. For example, a carboxyl structural element (COOH) interacts with sodium to form a sodium-carboxylate salt, the COO— being a residual structural element.

By “solvent(s)” is meant a substance in which various other substances may be fully or partially dissolved. In the present invention, preferred solvents include aqueous solvents, and solvents having a dielectric constant less than that of water. Preferred solvents having a dielectric constant less than that of water. The dielectric constant is a measure of the polarity of a solvent and dielectric constants for exemplary solvents are shown in Table 1.

TABLE 1
Characteristics of Exemplary Solvents

| | | Dielectric |
|---|---|---|
| Solvent | Boiling Pt., ° C. | constant |
| Water | 100 | 80 |
| Methanol | 68 | 33 |
| Ethanol | 78 | 24.3 |
| 1-propanol | 97 | 20.1 |
| 1-butanol | 118 | 17.8 |
| acetic acid | 118 | 6.15 |
| Acetone | 56 | 20.7 |
| methyl ethyl ketone | 80 | 18.5 |
| ethyl acetate | 78 | 6.02 |
| Acetonitrile | 81 | 36.6 |
| N,N-dimethylformamide | 153 | 38.3 |
| (DMF) | | |
| diemthyl sulfoxide (DMSO) | 189 | 47.2 |
| Hexane | 69 | 2.02 |
| Benzene | 80 | 2.28 |
| diethyl ether | 35 | 4.34 |
| tetrahydrofuran (THF) | 66 | 7.52 |
| methylene chloride | 40 | 9.08 |
| carbon tetrachloride | 76 | 2.24 |

The solvents water, methanol, ethanol, 1-propanol, 1-butanol, and acetic acid are polar protic solvents having a hydrogen atom attached to an electronegative atom, typically oxygen. The solvents acetone, ethyl acetate, methyl ethyl ketone, and acetonitrile are dipolar aprotic solvents, and are in one embodiment, preferred for use in forming the inventive complexes. Dipolar aprotic solvents do not contain an OH bond but typically have a large bond dipole by virtue of a multiple bond between carbon and either oxygen or nitrogen. Most dipolar aprotic solvents contain a C—O double bond. Solvents having a dielectric constant less than that of water are particularly useful in the formation of the inventive complexes. The dipolar aprotic solvents noted in Table 1 have a dielectric constant at least two-fold lower than water and a dipole moment close to or greater than water.

By “structural element” is meant a chemical group that (i) is part of a larger molecule, and (ii) possesses distinguishable chemical functionality. For example, an acidic group or a basic group on a compound is a structural element.

By “substance” is meant a chemical entity having specific characteristics.

By “tight-ion pair” is meant a pair of ions that are, at physiologic pH and in an aqueous environment are not readily interchangeable with other loosely paired or free ions that may be present in the environment of the tight-ion pair. A tight-ion pair can be experimentally detected by noting the absence of interchange of a member of a tight ion-pair with another ion, at physiologic pH and in an aqueous environment, using isotopic labeling and NMR or mass spectroscopy. Tight ion pairs also can be found experimentally by noting the lack of separation of the ion-pair, at physiologic pH and in an aqueous environment, using reverse phase HPLC.

By “therapeutically effective amount” is meant that amount of a drug that elicits the biological or medicinal response in a tissue system, animal or human that is being sought by a researcher, veterinarian, medical doctor or other clinician, which includes alleviation of the symptoms of the disease or disorder being treated. More specifically, a therapeutically effective amount of the inventive substances preferably alleviates symptoms, complications, or biochemical indicia of pain syndromes. The exact dose will be ascertainable by one skilled in the art using known techniques (see, e.g., Lieberman, Pharmaceutical Dosage Forms (Vols. 1-3, 1992); Lloyd, 1999, The Art, Science, and Technology of Pharmaceutical Compounding; and Pickar, 1999, Dosage Calculations). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compounds.

By “tramadol” is meant tramadol, its optical isomers, its metabolites, and pharmaceutically acceptably salts of any of the above. A preferred pharmaceutically acceptable salt of tramadol is tramadol HCl.

By “tramadol equivalent” is meant the weight of tramadol converted from a pharmaceutically acceptable salt back to the free base form. As the molecular weight is different for various salts of tramadol, it is confusing to report the dose for a dosage form according to the weight of the substance. It is preferred to report the dose as the tramadol equivalent, i.e. the weight equivalent of tramadol present in the salt. On this basis, certain embodiments according to the invention may comprise a tramadol equivalent present in the dosage form ranging from about 20 mg to about 500 mg, preferably from about 50 mg to about 400 mg, and more preferably from about 50 mg to about 300 mg. Particular dosage forms may contain about 20 mg, about 30 mg, about 40 mg, about 50 mg, about 100 mg, about 150 mg, about 200 mg, about 250 mg, about 300 mg, or about 400 mg weight equivalents in a given dosage form.

By “transport moiety” is meant a compound that is capable of forming, or a residue of that compound that has formed, a complex with a drug, wherein the transport moiety serves to improve transport of the drug across epithelial tissue, compared to that of the uncomplexed drug. The transport moiety comprises a hydrophobic portion and a(n) acidic, basic, or zwitterionic structural element, or a(n) acidic, basic, or zwitterionic residual structural element. In a preferred embodiment, the hydrophobic portion comprises a hydrocarbon chain. In an embodiment, the pKa of a basic structural element or basic residual structural element is greater than about 7.0, preferably greater than about 8.0. Zwitterionic structural elements or zwitterionic residual structural elements are analyzed in terms of their individual basic structural element or basic residual structural element or their acidic structural element or acidic residual structural element, depending upon how the complex with the drug moiety is to be formed.

In a more preferred embodiment, transport moieties comprise pharmaceutically acceptable acids, including but not limited to carboxylic acids, and salts thereof. In embodiments, transport moieties comprise fatty acids or its salts, benzenesulfonic acid or its salts, benzoic acid or its salts, fumaric acid or its salts, or salicylic acid or its salts. In preferred embodiments the fatty acids or their salts, comprise from 6 to 18 carbon atoms (C6-C18), more preferably 8 to 16 carbon atoms (C8-C16), even more preferably 10 to 14 carbon atoms (C10-C14), and most preferably 12 carbon atoms (C12).

In more preferred embodiments, transport moieties comprise alkyl sulfates (either saturated or unsaturated) and their salts, such as potassium, magnesium, and sodium salts, including particularly sodium octyl sulfate, sodium decyl sulfate, sodium lauryl sulfate, and sodium tetradecyl sulfate. In preferred embodiments the alkyl sulfate or its salt comprise from 6 to 18 carbon atoms (C6-C18), more preferably 8 to 16 carbon atoms (C8-C16), even more preferably 10 to 14 carbon atoms (C10-C14), and most preferably 12 carbon atoms (C12). Also suitable are other anionic surfactants.

In another more preferred embodiment, transport moieties comprise pharmaceutically acceptable primary amines or salts thereof, particularly primary aliphatic amines (both saturated and unsaturated) or salts thereof, diethanolamine, ethylenediamine, procaine, choline, tromethamine, meglumine, magnesium, aluminum, calcium, zinc, alkyltrimethylanmuonium hydroxides, alkyltrimethylammonium bromides, benzalkonium chloride and benzethonium chloride. Also useful are other pharmaceutically acceptable compounds that comprise secondary or tertiary amines, and their salts, and cationic surfactants.

By “upper gastrointestinal tract bioavailability of gabapentin” is meant the AUC_{inf} obtained when a dose of a substance comprising gabapentin is administered to the upper gastrointestinal tract divided by the AUC_{inf} obtained when a dose of a substance comprising gabapentin is administered intravenously.

By “upper gastrointestinal tract” or “upper G.I. tract” or “small intestine” is meant that portion of the gastrointestinal tract that includes the stomach, the duodenum, the jejunum, and/or the ileum.

By “window” is meant a period of time having a defined duration. Windows preferably begin at time of administration of a dosage form to a patient, or any time thereafter. For instance, in an embodiment a window may have a duration of about 12 hours. In a preferable embodiments, the window may begin at a variety of times. For instance, in a preferable embodiment, the window may begin about 1 hour after administration of a dosage form, and have a duration of about 12 hours, which means that the window would open about 1 hour after administration of the dosage from and close at about 13 hours following administration of the dosage form.

By “zero order rate of release” is meant a rate of release wherein the amount of drug released as a function of time is substantially constant. More particularly, the rate of release of drug as a function of time shall vary by less than about 30%, preferably, less than about 20%, more preferably, less than about 10%, most preferably, less than about 5%, wherein the measurement is taken over the period of time wherein the cumulative release is between about 25% and about 75%, preferably, between about 25% and about 90% by total weight of drug in the dosage form.

By “zero order plasma profile” is meant a substantially flat or unchanging amount of a particular drug in the plasma of a patient over a particular time interval. Generally, the plasma concentration of a drug exhibiting a zero order plasma profile will vary by no more than about 30% and preferably by no more than about 10% from one time interval to the subsequent time interval.

### II. IMPROVED NEUROPATHIC PAIN TREATMENTS

The inventors have unexpectedly discovered that it is possible to solve the problems in the art discussed above using substances, compositions, dosage forms and methods that deliver tramadol and gabapentin using controlled delivery approaches as set forth herein. Such substances, compositions, dosage forms and methods are useful, inter alia, in the treatment of neuropathic pain, and possibly also other forms of pain.

Single agent treatment of neuropathic pain using the anticonvulsant gabapentin, specifically for the treatment of painful diabetic neuropathy and postherpetic neuralgia, has been demonstrated (Wheeler, G., Curr. Opin. Invest. Drugs, 3(3):470 (2002)). While the effective dose of this agent is quite large (e.g. around 600 mg tid (1800 mg total/day) suggested for gabapentin), it seems to have relatively benign side effects.

Tramadol is also thought to be useful as a single agent in the treatment of neuropathic pain. Harati, Y. et al, Neurology 50(6) 1842-6 (1998), Sindrup S. H. et al, Pain 83(1) 85-90 (1999). Tramadol has a number of side effects, including nausea and vomiting, that can limit the dose administrable to patients. This limitation thus controls the maximum dose of tramadol administrable to patients in pain, and therefore may reduce the therapeutic effect provided by tramadol. Typical daily dosages of tramadol for neuropathic pain range from 100 to 400 mg.

Therefore, daily doses of both gabapentin and tramadol for neuropathic pain would seem to require dosing a patient with at least about 2000 milligrams of drug substance per day in order to achieve effective neuropathic pain relief. If, as suggested above, these drugs were administered as a qd oral dosage form, this would require a patient to swallow more than 2000 milligrams of drug plus formulation all at once. This regimen would likely have extremely poor compliance.

The inventors have recognized that several technical insights can be combined into a model dosing regimen that provides for neuropathic pain relief, qd dosing, and significantly reduced volumes or mass of drug to be administered. The inventive oral dosage forms therefore would be more palatable to patients, leading to increased compliance.

The three primary technical insights are: (1) pharmacodynamic synergy between gabapentin and tramadol at certain ratios; (2) the need for substances that comprise gabapentin wherein such substances exhibit improved absorption, preferably colonic absorption, of gabapentin; and (3) that certain pharmacokinetic principles can be applied to model potential pharmacodynamic effects.

The pharmacodynamic synergy between gabapentin and tramadol at certain ratios was first articulated in U.S. Pat. No. 6,562,865 to Codd et al. (“Codd”). Codd discloses synergy between gabapentin and tramadol in a non-clinical model of neuropathic pain. That is, in certain combinations, gabapentin and tramadol can be administered together to achieve effective pain relief in doses less than what would be required if administered as single agents. These synergies may be harnessed to improve the therapeutic index of tramadol and gabapentin with respect to neuropathic pain and possibly other forms of pain as well. In effect, harnessing the teachings of Codd permits the amount of tramadol and substances comprising gabapentin to be reduced significantly.

However, the inventors recognized a problem with adapting the teachings of Codd to qd dosage forms. Qd dosing implies that the drugs in question can be reasonably well absorbed in the colon, since the drugs will move through the GI tract during the course of the day. If a drug is poorly colonically absorbed, then achieving the synergies taught by Codd will be difficult to achieve.

The inventors have recognized that gabapentin is poorly absorbed in the lower G.I. tract, and possibly even in portions of the upper G.I. tract. This is borne out by the understanding in the art that gabapentin is absorbed from the proximal small intestine into the blood stream by the L-amino acid transport system (Johannessen, supra at 350). Bioavailability of the drug is dose dependent, apparently because the L-amino acid transport system saturates, limiting the amount of drug absorbed (Stewart, B.H. et al., Pharm. Res., 10:276 (1993)). For example, serum gabapentin concentrations increase linearly with doses up to about 1800 mg/d, and then continue to increase at higher doses but less than expected, possibly because the absorption mechanism from the upper G.I. tract becomes saturated (Stewart, supra.). The L-amino transport system responsible for absorption of gabapentin is present primarily in the epithelial cells of the small intestine (Kanai, Y. et al., J. Toxicol. Sci., 28(1):1 (2003)), thus limiting the absorption of the drug.

In contrast, the absorption of tramadol from colon is similar to that from the upper GI-tract based on the equivalent bioavailability for a sustained-release dosage form and an immediate-release capsule of tramadol (Malonne, H. et al Br. J. Clin. Pharmacol. 57(3) 270-8 (2003)).

Accordingly, while tramadol might be usefully administered using conventional controlled release technologies, gabapentin cannot. The inventors have recognized that poor lower G.I. tract (and portions of the distal upper G.I. tract) absorption implies that conventional controlled release (CR) techniques will not work in the development of an qd oral dosage form that comprises gabapentin. Generally, a CR dosage form will move through the upper G.I. tract to the lower G.I. tract within 8-10 hours or less. Once the dosage form that included tramadol and gabapentin arrived at the lower G.I. tract, absorption of the compound would be significantly reduced. In fact, absorption from an IR dosage form may be essentially complete as quickly as 4 hours after dosing. Therefore, CR dosage forms would have to be dosed more frequently than bid and definitely more frequently than qd to maintain efficacy throughout the day. Such frequent dosing, as noted elsewhere herein, is undesirable in pain control.

Accordingly, the inventors have surprisingly recognized that only a specific sub-class of controlled release technologies, referred to herein as controlled delivery technologies, would suffice to provide bid or qd dosing of tramadol and gabapentin.

These controlled delivery technologies comprise substances comprising gabapentin. In a preferred embodiment, gabapentin in the form of an alkyl sulfate complex is controllably delivered to a patient in need thereof. In other embodiments, controlled delivery technologies comprise technologies that selectively deliver tramadol and gabapentin to portions of the upper GI that demonstrate clinically acceptable absorption of tramadol and gabapentin. An example of such an embodiment is a gastric retention dosage form. In other embodiments of the present invention, there is the proviso that the substance that comprises gabapentin excludes gabapentin prodrugs wherein the gabapentin prodrug comprises chemical structure that enhances colonic absorption of the gabapentin prodrug as compared to gabapentin.

The next step taken by the inventors was to recognize that the pharmacodynamic relationships that exist in the rats models used in the work of Codd may not hold true when extended to controlled delivery technologies and/or to other species. Accordingly, the inventors modeled the expected metabolism of tramadol (gabapentin is not metabolized extensively in most species of interest), in order to develop a controlled delivery dosage form that may provide the synergies noted in Codd. In particular, the inventors selected certain tramadol isomers and metabolites for inclusion in the model, to provide appropriate results.

The following tramadol pharmacokinetic parameters have been reported (Liu et al. 2003) after the oral administration of a single dose of 10 mg/kg tramadol HCl to 8 male Sprague-Dawley rats:

| | Tₘₐₓ (min) | Cₘₐₓ (μg/L) | AUC_{inf} (μg L⁻¹ min) |
|---|---|---|---|
| (+)-trans-T | 22 | 193 | 21501 |
| (−)-trans-T | 22 | 74 | 5257 |
| Total | | 267 | 26758 |
| (+) M1 | 34 (21) | 136 (32) | 16242 (2648) |
| (−) M1 | 41 (24) | 195 (63) | 21178 (1987) |
| Total | | 331 | 31420 |

M1 = trans-O-demethyltramadol

The following gabapentin pharmacokinetic parameters have been reported (Cundy et al. 2004) after the oral administration of a single dose of 25 mg/kg gabapentin to 6 male Sprague-Dawley rats:

| | Tₘₐₓ (h) | Cₘₐₓ (μg/mL) | AUC_{inf} (μg * h/mL) |
|---|---|---|---|
| Gabapentin | 1.8 | 6.85 | 32.8 |

Assuming no significant drug-to-drug pharmacokinetic interaction, the expected pharmacokinetic parameters after a single administration of 10 mg/kg tramadol HCl and 5 mg/kg gabapentin are shown below:

| | Cₘₐₓ (μg/mL) | AUC_{inf} (μg * h/mL) |
|---|---|---|
| (±) tramadol | 0.267 | 0.446 |
| (±) M1 | 0.331 | 0.523 |
| gabapentin | 1.37 | 6.56 |

An average dose for treating neuropathic pain was 210 mg per day (Harati et al. 1998) while in another study the individual dose was titrated between 200 and 400 mg per day (Sindrup et al. 1999). As part of a preferred embodiment, human pharmacokinetic data have been reviewed/summarized (Grond and Sablotzki 2004). Tramadol pharmacokinetics was studied mostly at or below 200 mg. In one pharmacokinetic study when tramadol 400 mg was given, the pharmacokinetic parameters, Cₘₐₓ and AUC_{inf} appeared to be proportional to the dose. After five days of dosing tramadol 100 mg twice daily, the Cₘₐₓ was 414 μg/L and AUC₀₋₁₂ was 2970 μg*h/L at steady state. In a study of tramadol immediate-release capsule (50 mg given four times daily) and a new modified release formulation of tramadol (200 mg given once daily)(Malonne et al. 2004), the steady-state pharmacokinetics of both (+) and (−) tramadol have been reported:

| | Immediate Release Capsule | | | | | |
|---|---|---|---|---|---|---|
| | Cₘₐₓ | | | Modified Release Formulation | | |
| | (ng/ | Cₘᵢₙ | AUC₀₋₂₄ | Cₘₐₓ | Cₘᵢₙ | AUC₀₋₂₄ |
| | mL) | (ng/mL) | (ng * h/mL) | (ng/mL) | (ng/mL) | (ng * h/ml) |
| (+) | 190 | 111 | 3429 | 239 (81) | 81 (36) | 3763 (1275) |
| tramadol | (56) | (48) | (1260) | | | |
| (−) | 157 | 86 | 2719 | 199 (75) | 62 (30) | 3005 (1120) |
| tramadol | (51) | (42) | (1093) | | | |
| (+) M1 | 36 | 21 | 644 | 42 (18.4) | 16.71 (10.41) | 704.12 (327.09) |
| | (13) | (10) | (231) | | | |
| (−) M1 | 43 | 22 | 690 | 46 (18) | 16 (8) | 745 (286) |
| | (14) | (10) | (191) | | | |

The pharmacokinetics for orally administered gabapentin are not linear(Gidal et al. 1998). The amount of gabapentin absorbed is related to the oral dose as described below:

$$F \cdot Dose = \frac{2720 mg}{4080 mg + Dose}$$

where F is the fraction of the dose absorbed and Dose is the amount of gabapentin administered orally given q8h.

In the Chung model disclosed in Codd, the ED50 is 94.47 and 439.50 mg/kg for tramadol HCl and gabapentin, respectively. For humans, an average therapeutic dose for treating neuropathic pain is 200 and 1800 mg/day for tramadol and gabapentin, respectively. Accordingly, tramadol is more potent than gabapentin in both rats and humans, although in a different ratio. The finding of a beneficial 0.9:0.1 ED50 value ratio for Chung model is a 0.52 gabapentin to tramadol mass ratio, or 0.455 gabapentin to tramadol HCl mass ratio. Therefore, the inventors have modeled a modification of the mass ratio of Codd to include a relative inter-species potency term that modifies the relative mass of tramadol equivalents and gabapentin equivalents as reported by Codd. The inventors have selected a range of relative potencies that play into the selection of the inventive weight ratios.

In another modeling approach, the dose of gabapentin for humans can be evaluated in order to match the AUC_{inf} ratio for gabapentin to tramadol and O-demthyltramadol in rat. Per Codd's disclosure regarding the Chung model (i.e. rat), AUC_{inf} for gabapentin is 6.77 fold that for (±) tramadol and (±) O-demthyltramadol. In the case of 200 mg tramadol per day for humans, the following pharmacokinetic parameters are expected:

| | (+) tramadol | (−) tramadol | (+) M1 | (−) M1 | Gabapentin |
|---|---|---|---|---|---|
| Cₘₐₓ | 0.190 | 0.157 | 0.036 | 0.043 | |
| (μg/mL) | | | | | |
| C_{avg} (μg/ | 0.143 | 0.113 | 0.011 | 0.012 | 1.89 |
| mL) | | | | | |
| Cₘᵢₙ | 0.111 | 0.086 | 0.021 | 0.022 | |
| (μg/mL) | | | | | |

It requires a daily dose of 505 mg gabapentin equivalent to achieve an average plasma gabapentin concentration of 1.89 μg/mL, based on reports of gabapentin bioavailability in humans. Additional ranges can be determined for other species.

Using the above techniques, the inventors have arrived at novel and nonobvious daily dosage ranges that provide average concentrations that are based on the synergies of Codd and provide for reduced amount of tramadol and substances comprising gabapentin needed to treat patients. In an oral dosage form according to the invention, the weight ratio of gabapentin equivalent to tramadol equivalent present in the oral dosage form ranges from about 0.75:1 to about 6.5:1. More preferably, the weight ratio of gabapentin equivalent to tramadol equivalent present in the oral dosage form ranges from 0.80:1 to 5.5:1, still more preferably from about 0.90:1 to about 4.5:1. In a preferred embodiment, the total weight of the tramadol and substance that comprises gabapentin present in the oral dosage form is less than about 1500 milligrams, more preferably less than about 1000 milligrams, and still more preferably less than about 750 milligrams. This reduced volumes of tramadol and substances that comprise gabapentin provides the advantages discussed elsewhere herein, such as (but not limited to) improved compliance due to a lower volume the dosage forms that need to be swallowed.

### REFERENCES USED IN THE MODEL INCLUDE

Cundy, K. C., T. Annamalai, L. Bu, J. De Vera, J. Estrela, W. Luo, P. Shirsat, A. Tomeros, F. Yao, J. Zou, R. W. Barrett and M. A. Gallop (2004). “YXP13512 [(±)-1-([(-Isobutanoyloxyethoxy)carbonyl]aminomethyl)-1-cyclohexane Acetic Acid], A Novel Gabapentin Prodrug: II. Improved Oral Bioavailability, Dose Proportionality, and Colonic Absorption Compared with Gabapentin in Rats and Monkeys.” Journal of Pharmacology and Experimental Therapeutics 311(1): 324-33.
Gidal, B. E., J. DeCerce, H. N. Bockbrader, J. Gonzalez, S. Kruger, M. E. Pitterle, P. Rutecki and R. E. Ramsay (1998). “Gabapentin Bioavailability: Effect of Dose and Frequency of Administration in Adult Patients with Epilepsy.” Epilepsy Research 31(2): 91-9.
Grond, S. and A. Sablotzki (2004). “Clinical Pharmacology of Tramadol.” Clinical Pharmacokinetics 43(13): 879-923.
Harati, Y., C. Gooch, M. Swenson, S. Edelman, D. Greene, P. Raskin, P. Donofrio, D. Comblath, R. Sachdeo, C. Siu and M. Kamin (1998). “Double-Blind Randomized Trial of Tramadol for the Treatment of the Pain of Diabetic Neuropathy.” Neurology 50(6): 1842-6.
Liu, H.-C., S.-M. Jin and Y.-L. Wang (2003). “Gender-related differences in pharmacokinetics of enantiomers of trans-tramadol and its active metabolite, trans-O-demethyltramadol, in rats.” Acta Pharmacologica Sinica 24(12): 1265-9.
Malonne, H., B. Sonet, B. Streel, S. Lebrun, S. D. Niet, A. Sereno and F. Vanderbist (2004). “Pharmacokinetic Evaluation of a New Oral Sustained Release Dosage Form of Tramadol.” British Journal of Clinical Pharmacology 57(3): 270-8.
Sindrup, S. H., G. Andersen, C. Madsen, T. Smith, K. Brosen and T. S. Jensen (1999). “Tramadol Relieves Pain and Allodynia in Polyneuropathy: a Randomised, Double-Blind, Controlled Trial.” Pain 83(1): 85-90.

Various embodiments of the inventive controlled delivery technologies will now be discussed further herein.

III. CONTROLLED DELIVERY. COMPLEX FORMATION AND CHARACTERIZATION

In certain embodiments, gabapentin is modified so as to demonstrate improved lower G.I. tract absorption. Pharmaceutical development typically targets drug forms for absorption in the upper G.I. tract instead of the lower G.I. tract because the upper G.I. tract has a far greater surface area for absorption of drugs than does the lower G.I. tract. The lower G.I. tract lacks microvilli which are present in the upper G.I. tract. The presence of microvilli greatly increases the surface area for drug absorption, and the upper G.I. tract has 480 times the surface area than does the lower G.I. tract. Differences in the cellular characteristics of the upper and lower G.I. tracts also contribute to the poor absorption of molecules in the lower G.I. tract.

To provide constant dosing treatments, conventional pharmaceutical development has suggested various controlled release drug systems. Such systems function by releasing their payload of drugs over an extended period of time following administration. However, these conventional forms of controlled release systems are not effective in the case of drugs exhibiting minimal colonic absorption. Since the drugs are only absorbed in the upper G.I. tract and since the residence time of the drug in the upper G.I. tract is only four to six hours, the fact that a proposed controlled release dosage form may release its payload after the residence period of the dosage form in the upper G.I. does not mean the that body will continue to absorb the controlled release drug past the four to six hours of upper G.I. tract residence. Instead, the drug released by the controlled release dosage form after the dosage form has entered the lower G.I. tract is generally not absorbed and, instead, is expelled from the body.

It has been surprisingly found that many common drug moieties with poor absorption characteristics, once complexed with certain transport moieties, exhibit significantly enhanced absorption, particularly lower G.I. tract absorption although upper G.I. tract absorption may also be enhanced. It is further surprising that complexes, such as certain substances comprising gabapentin, according to the invention show improved absorption as compared to loose ion-pairs (i.e. a non-complexed form) that comprise the same ions as the inventive complexes.

These unexpected results have been found to apply to many categories of drug moieties, including drug moieties that comprise a basic structural element or a basic residual structural element. The unexpected results of the present invention also apply to drug moieties that comprise a zwitterionic structural element or a zwitterionic residual structural element. An example of such a drug moiety comprises gabapentin, disclosed generally in U.S. patent application Ser. No. 10/978,136 filed Oct. 29,2004.

While not wishing to be bound by specific understanding of mechanisms, the inventors reason as follows:

When loose ion-pairs are placed in a polar solvent environment, it is assumed that polar solvent molecules will insert themselves in the space occupied by the ionic bond, thus driving apart the bound ions. A solvation shell, comprising polar solvent molecules electrostatically bonded to a free ion, may be formed around the free ion. This solvation shell then prevents the free ion from forming anything but a loose ion-pairing ionic bond with another free ion. In a situation wherein there are multiple types of counter ions present in the polar solvent, any given loose ion-pairing may be relatively susceptible to counter-ion competition.

This effect is more pronounced as the polarity, expressed as the dielectric constant of the solvent, increases. Based on Coulomb's law, the force between two ions with charges (q1) and (q2) and separated by a distance(r) in a medium of dielectric constant (e) is:

$$F = - \frac{{q}_{1} {q}_{2}}{4 π {ɛ}_{0} ɛ {r}^{2}} \qquad ( Equation 2 )$$

where ε₀ is the constant of permittivity of space. The equation shows the importance of dielectric constant (ε) on the stability of a loose ion-pair in solution. In aqueous solution that has a high dielectric constant (ε=80), the electrostatic attraction force is significantly reduced if water molecules attack the ionic bonding and separate the opposite charged ions.

Therefore, high dielectric constant solvent molecules, once present in the vicinity of the ionic bond, will attack the bond and eventually break it. The unbound ions then are free to move around in the solvent. These properties characterize a loose ion-pair.

Tight ion-pairs are formed differently from loose-ion pairs, and consequently possess different properties from a loose ion-pair. Tight ion-pairs are formed by reducing the number of polar solvent molecules in the bond space between two ions. This allows the ions to move tightly together, and results in a bond that is significantly stronger than a loose ion-pair bond, but is still considered an ionic bond. As disclosed more fully herein, tight ion-pairs are obtained using less polar solvents than water so as to reduce entrapment of polar solvents between the ions.

For additional discussion of loose and tight ion-pairs, see D. Quintanar-Guerrero et al., “Applications of the Ion Pair Concept to Hydrophilic Substances with Special Emphasis on Peptides,” Pharm. Res. 14(2):119-127 (1997).

The difference between loose and tight ion-pairing also can be observed using chromatographic methods. Using reverse phase chromatography, loose ion-pairs can be readily separated under conditions that will not separate tight ion-pairs.

Bonds according to this invention may also be made stronger by selecting the strength of the cation and anion relative to one another. For instance, in the case where the solvent is water, the cation (base) and anion (acid) can be selected to attract one another more strongly. If a weaker bond is desired, then weaker attraction may be selected.

Portions of biological membranes can be modeled to a first order approximation as lipid bilayers for purposes of understanding molecular transport across such membranes. Transport across the lipid bilayer portions (as opposed to active transporters, etc.) is unfavorable for ions because of unfavorable portioning. Various researchers have proposed that charge neutralization of such ions can enhance cross-membrane transport.

In the “ion-pair” theory, ionic drug moieties are paired with transport moiety counter ions to “bury” the charge and render the resulting ion-pair more liable to move through a lipid bilayer. This approach has generated a fair amount of attention and research, especially with regards to enhancing absorption of orally administered drugs across the intestinal epithelium.

While ion-pairing has generated a lot of attention and research, it has not always generated a lot of success. For instance, ion-pairs of two antiviral compounds were found not to result in increased absorption due to the effects of the ion-pair on trans-cellular transport, but rather to an effect on monolayer integrity. The authors concluded that the formation of ion pairs may not be very efficient as a strategy to enhance transepithelial transport of charged hydrophilic compounds as competition by other ions found in in vivo systems may abolish the beneficial effect of counter-ions. J. Van Gelder et al., “Evaluation of the Potential of Ion Pair Formation to Improve the Oral Absorption of two Potent Antiviral Compounds, AMD3100 and PMPA”, Int. J. of Pharmaceutics 186:127-136 (1999). Other authors have noted that absorption experiments with ion-pairs have not always pointed at clear-cut mechanisms. D. Quintanar-Guerrero et al., Applications of the Ion Pair Concept to Hydrophilic Substances with Special Emphasis on Peptides, Pharm. Res. 14(2):119-127 (1997).

The inventors have unexpectedly discovered that a problem with these ion-pair absorption experiments is that they were performed using loose-ion pairs, rather than tight ion-pairs. Indeed, many ion-pair absorption experiments disclosed in the art do not even expressly differentiate between loose ion-pairs and tight ion-pairs. One of skill has to distinguish that loose ion-pairs are disclosed by actually reviewing the disclosed methods of making the ion-pairs and noting that such disclosed methods of making are directed to loose ion-pairs not tight ion-pairs. Loose ion-pairs are relatively susceptible to counter-ion competition, and to solvent-mediated (e.g. water-mediated) cleavage of the ionic bonds that bind loose ion-pairs. Accordingly, when the drug moiety of the ion-pair arrives at an intestinal epithelial cell membrane wall, it may or may not be associated in a loose ion-pair with a transport moiety. The chances of the ion-pair existing near the membrane wall may depend more on the local concentration. of the two individual ions than on the ion bond keeping the ions together. Absent the two moieties being bound when they approached an intestinal epithelial cell membrane wall, the rate of absorption of the non-complexed drug moiety might be unaffected by the non-complexed transport moiety. Therefore, loose ion-pairs might have only a limited impact on absorption compared to administration of the drug moiety alone.

In contrast, the inventive complexes possess bonds that are more stable in the presence of polar solvents such as water. Accordingly, the inventors reasoned that, by forming a complex, the drug moiety and the transport moiety would be more likely to be associated as ion-pairs at the time that the moieties would be near the membrane wall. This association would increase the chances that the charges of the moieties would be buried and render the resulting ion-pair more liable to move through the cell membrane.

In an embodiment, the complex comprises a tight ion-pair bond between the drug moiety and the transport moiety. As discussed herein, tight ion-pair bonds are more stable than loose ion-pair bonds, thus increasing the likelihood that the drug moiety and the transport moiety would be associated as ion-pairs at the time that the moieties would be near the membrane wall. This association would increase the chances that the charges of the moieties would be buried and render the tight ion-pair bound complex more liable to move through the cell membrane.

It should be noted that the inventive complexes may improve absorption relative to the non-complexed drug moiety throughout the G.I. tract, not just the lower G.I. tract, as the complex is intended to improve transcellular transport generally, not just in the lower G.I. tract. For instance, if the drug moiety is a substrate for an active transporter found primarily in the upper G.I., the complex formed from the drug moiety may still be a substrate for that transporter. Accordingly, the total transport may be a sum of the transport flux effected by the transporter plus the improved transcellular transport provided by the present invention. In an embodiment, the inventive complex provides improved absorption in the upper G.I. tract, the lower G.I. tract, and both the upper G.I. tract and the lower G.I. tract.

Complexes according to the invention can be made up of a variety of drug and transport moieties. Generally speaking, the drug moiety is selected first, and then the appropriate transport moiety is selected to form the inventive complex. One of skill could consider a number of factors in selecting transport moieties, including but not limited to the toxicity and tolerability of the transport moiety, the polarity of the structural element or structural element residue of the drug moiety, the strength of the structural element or structural element residue of the drug moiety, the strength of the structural element or structural element residue of the transport moiety, possible therapeutic advantages of the transport moiety. In certain preferred embodiments, the hydrophobic portions of the transport moiety comprises a hydrophobic chain, more preferably an alkyl chain. This alkyl chain may help to promote stability of the complex through sterically protecting the ionic bond from attack by polar solvent molecules.

In preferred embodiments the transport moieties comprise alkyl sulfates or their salts, having from 6 to 18 carbon atoms (C6-C18), more preferably 8 to 16 carbon atoms (C8-C16), even more preferably 10 to 14 carbon atoms (C10-C14), and most preferably 12 carbon atoms (C12). In other preferred embodiments, the transport moieties comprise fatty acids, or their salts, having from 6 to 18 carbon atoms (C6-C18), more preferably 8 to 16 carbon atoms (C8-C16), even more preferably 10 to 14 carbon atoms (C10-C14), and most preferably 12 carbon atoms (C12). Methods of making the inventive gabapentin complexes are disclosed herein, including the appended Examples.

An alternative manner of improving lower G.I. absorption of gabapentin is to produce prodrugs of the compounds that are substrates for active transporters expressed in epithelial cells lining the lumen of the human colon. United States Patent Application 20030158254 to Zerangue et al., filed Aug. 21, 2003, entitled “Engineering absorption of therapeutic compounds via colonic transporters” (“Zerangue”), discloses drugs modified to be such substrates, including compounds suitable for use in extended release oral dosage forms, particularly those that release drug over periods of greater than about 2-4 hours following administration.

Zerangue discloses a variety of transporters useful in the practice of this invention, comprising the sodium dependent multi-vitamin transporter (SMVT), and monocarboxylate transporters 1 and 4 (MCT 1 and MCT 4). Zerangue also discloses methods of identifying agents or conjugate moieties that are substrates of a transporter, and agents, conjugates, and conjugate moieties that can be screened. In particular, Zerangue discloses compounds to be screened that are variants of known transporter substrates. Such compounds comprise bile salts or acids, steroids, ecosanoids, or natural toxins or analogs thereof, as described by Smith, Am. J. Physiol. 2230, 974-978 (1987); Smith, Am. J. Physiol. 252, G479-G484 (1993); Boyer, Proc. Natl. Acad. Sci. USA 90, 435-438 (1993); Fricker, Biochem. J. 299, 665-670 (1994); Ficker, Biochem J. 299, 665-670 (1994); Ballatori, Am. J. Physiol. 278. Zerangue further discloses the linkage of agents to conjugate moieties, and several prodrugs, comprising pivaloxymethyl gabaptentin carbamate, gabapentin acetoxyethyl carbamate, and alpha-aminopropylisobutyryl gabapentin. Other gabapentin prodrugs useful in the practice of this invention are disclosed at KC Cundy et al., “XP13512 [(±)-1-([(alpha-isobutanoyloxyethoxy)carbonyl]aminomethyl)-1-cyclohexane acetic acid], a novel gabapentin prodrug: I. Design, synthesis, enzymatic conversion to gabapentin, and transport by intestinal solute transporters.” J Pharmacol Exp Ther. 2004 Oct;311(1):315-23, and KC Cundy et al., “XP13512 [(±)-1-([(alpha-isobutanoyloxyethoxy)carbonyl]aminomethyl)-1-cyclohexane acetic acid], a novel gabapentin prodrug: II. Improved oral bioavailability, dose proportionality, and colonic absorption compared with gabapentin in rats and monkeys.” J Pharmacol Exp Ther. 2004 October;311(1):324-33.

In other embodiments of the present invention, there is the proviso that the substances or complexes that comprise gabapentin excludes gabapentin prodrugs wherein the gabapentin prodrug comprises chemical structure that enhances colonic absorption of the gabapentin prodrug as compared to gabapentin.

In embodiments according to the invention, the inventive oral controlled delivery dosing structure is adapted to controllably deliver the substance that comprises gabapentin at a rate that is effective to, after a single administration of the oral dosage form to a patient, maintain a gabapentin plasma drug concentration that is at least about twenty-five percent of a gabapentin Cmax throughout a window of at least about fifteen hours duration after a time at which the gabapentin Cmax occurs. In preferable embodiments, the gabapentin plasma drug concentration is at least about thirty percent of a gabapentin Cmax throughout the window; more preferably the gabapentin plasma drug concentration is at least about thirty-five percent of a gabapentin Cmax throughout the window. In other preferable embodiments, the window is of at least about eighteen hours duration after a time at which the gabapentin Cmax occurs, more preferably the window is of at least about twenty hours duration after a time at which the gabapentin Cmax occurs.

IV. EXEMPLARY DOSAGE FORMS AND METHODS OF USE

A variety of dosage forms are suitable for use in the invention. A dosage form may be configured and formulated according to any design that delivers a desired dose of tramadol and substances that comprise gabapentin. In certain embodiments, the dosage form is orally administrable and is sized and shaped as a conventional tablet or capsule. Orally administrable dosage forms may be manufactured according to one of various different approaches. For example, the dosage form may be manufactured as a diffusion system, such as a reservoir device or matrix device, a dissolution system, such as encapsulated dissolution systems (including, for example, “tiny time pills”, and beads) and matrix dissolution systems, and combination diffusion/dissolution systems and ion-exchange resin systems, as described in Remington's Pharmaceutical Sciences, 18th Ed., pp. 1682-1685 (1990).

One important consideration in the practice of this invention is the physical state of the drug substance to be delivered by the dosage form. In certain embodiments, substances comprising gabapentin may be in a paste or liquid state, in which case solid dosage forms may not be suitable for use in the practice of this invention. In such cases, dosage forms capable of delivering substances in a paste or liquid state should be used. Alternatively, in certain embodiments, a different transport moiety may be used to raise the melting point of the substances, thus making it more likely that the inventive complexes will be present in a solid form.

A specific example of a dosage form suitable for use with the present invention is an osmotic dosage form. Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. An advantage to osmotic systems is that their operation is pH-independent and, thus, continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release, 35:1-21 (1995). Osmotic dosage forms are also described in detail in the following U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

The present invention provides a controlled delivery liquid formulation of tramadol and substances that comprise gabapentin for use with oral osmotic devices. Oral osmotic devices for delivering liquid formulations and methods of using them are known in the art, for example, as described and claimed in the following U.S. Patents owned by ALZA corporation: U.S. Pat. Nos. 6,419,952; 6,174,547; 6,551,613; 5,324,280; 4,111,201; and 6,174,547. Methods of using oral osmotic devices for delivering therapeutic agents at an ascending rate of release can be found in International Application Numbers: WO 98/06380, WO 98/23263, and WO 99/62496.

Exemplary liquid carriers for the present invention include lipophilic solvents (e.g., oils and lipids), surfactants, and hydrophilic solvents. Exemplary lipophilic solvents, for example, include, but are not limited to, Capmul PG-8, Caprol MPGO, Capryol 90, Plurol Oleique CC497, Capmul MCM, Labrafac PG, N-Decyl Alcohol, Caprol 10G100, Oleic Acid,Vitamin E, Maisine 35-1, Gelucire 33/01, Gelucire 44/14, Lauryl Alcohol, Captex 355EP, Captex 500, Capylic/Caplic Triglyceride, Peceol, Caprol ET, Labrafil M2125 CS, Labrafac CC, Labrafil M 1944 CS, Captex 8277, Myvacet 9-45, Isopropyl Nyristate, Caprol PGE 860, Olive Oil, Plurol Oleique, Peanut Oil, Captex 300 Low C6, and Capric Acid. Exemplary surfactants for example, include, but are not limited to, Vitamin E TPGS, Cremophor EL-P, Labrasol, Tween 20, Cremophor RH40, Pluronic L-121, Acconon S-35, Pluronic L-31, Pluronic L-35, Pluronic L-44, Tween 80, Pluronic L-64, Solutol HS-15, Span 20, Cremophor EL, Span 80, Pluronic L-43, and Tween 60. Exemplary hydrophilic solvents for example, include, but are not limited to, Isosorbide Dimethyl Ether, Polyethylene Glycol 400 (PEG-3000), Transcutol HP, Polyethylene Glycol 400 (PEG-4000), Polyethylene Glycol 400 (PEG-300), Polyethylene Glycol 400 (PEG-6000), Polyethylene Glycol 400 (PEG-400), Polyethylene Glycol 400 (PEG-8000), Polyethylene Glycol 400 (PEG-600), and Propylene Glycol (PG).

The skilled practitioner will understand that any formulation comprising a sufficient dosage of tramadol and substances comprising gabapentin solubilized in a liquid carrier suitable for administration to a subject and for use in an osmotic oral dosage form can be used in the present invention. In one exemplary embodiment of the present invention, the liquid carrier is PG, Solutol, Cremophor EL, or a combination thereof.

The liquid formulation according to the present invention can also comprise, for example, additional excipients such as an antioxidant, permeation enhancer and the like. Antioxidants can be provided to slow or effectively stop the rate of any autoxidizable material present in the capsule. Representative antioxidants can comprise a member selected from the group of ascorbic acid; alpha tocopherol; ascorbyl palmitate; ascorbates; isoascorbates; butylated hydroxyanisole; butylated hydroxytoluene; nordihydroguiaretic acid; esters of garlic acid comprising at least 3 carbon atoms comprising a member selected from the group consisting of propyl gallate, octyl gallate, decyl gallate, decyl gallate; 6-ethoxy-2,2,4-trimethyl-1,2-dihydro-guinoline; N-acetyl-2,6-di-t-butyl-p-aminophenol; butyl tyrosine; 3-tertiarybutyl-4-hydroxyanisole; 2-tertiary-butyl-4-hydroxyanisole; 4-chloro-2,6-ditertiary butyl phenol; 2,6-ditertiary butyl p-methoxy phenol; 2,6-ditertiary butyl-p-cresol: polymeric antioxidants; trihydroxybutyro-phenone physiologically acceptable salts of ascorbic acid, erythorbic acid, and ascorbyl acetate; calcium ascorbate; sodium ascorbate; sodium bisulfite; and the like. The amount of antioxidant used for the present purposes, for example, can be about 0.001% to 25% of the total weight of the composition present in the lumen. Antioxidants are known to the prior art in U.S. Pat. Nos. 2,707,154; 3,573,936; 3,637,772; 4,038,434; 4,186,465 and 4,559,237.

The inventive liquid formulation can comprise permeation enhancers that facilitate absorption of the active agent in the environment of use. Such enhancers can, for example, open the so-called “tight junctions” in the gastrointestinal tract or modify the effect of cellular components, such a p-glycoprotein and the like. Suitable enhancers can include alkali metal salts of salicyclic acid, such as sodium salicylate, caprylic or capric acid, such as sodium caprylate or sodium caprate, and the like. Enhancers can include, for example, the bile salts, such as sodium deoxycholate. Various p-glycoprotein modulators are described in U.S. Pat. Nos. 5,112,817 and 5,643,909. Various other absorption enhancing compounds and materials are described in U.S. Pat. No. 5,824,638. Enhancers can be used either alone or as mixtures in combination with other enhancers.

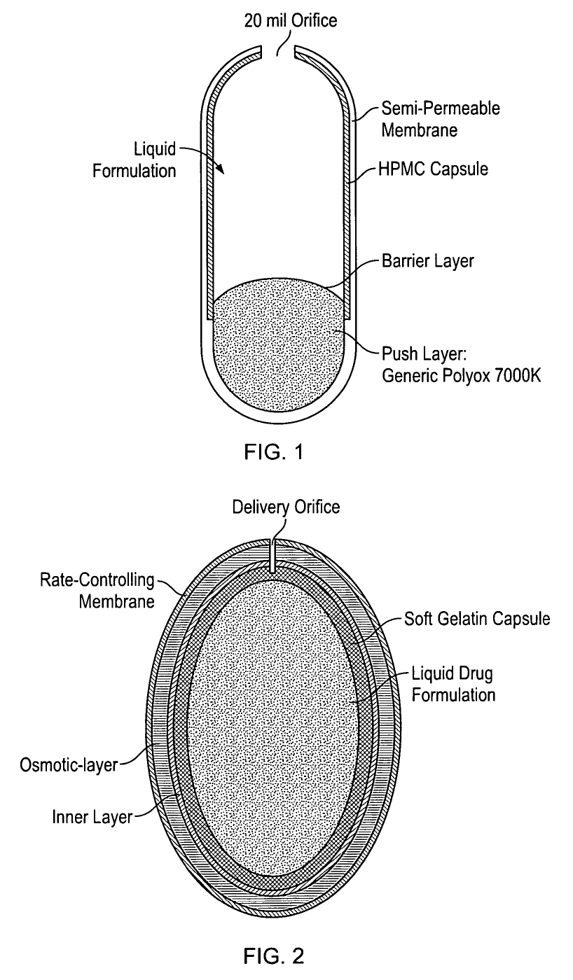

The osmotic dosage forms of the present invention can possess two distinct forms, a hard capsule form (shown in FIG.1), and a soft capsule form (shown in FIG. 2). The soft capsule, as used by the present invention, preferably in its final form comprises one piece. The one-piece capsule is of a sealed construction encapsulating the drug formulation therein. The capsule can be made by various processes including the plate process, the rotary die process, the reciprocating die process, and the continuous process. An example of the plate process is as follows. The plate process uses a set of molds. A warm sheet of a prepared capsule lamina-forming material is laid over the lower mold and the formulation poured on it. A second sheet of the lamina-forming material is placed over the formulation followed by the top mold. The mold set is placed under a press and a pressure applied, with or without heat, to form a unit capsule. The capsules are washed with a solvent for removing excess agent formulation from the exterior of the capsule, and the air-dried capsule is encapsulated with a semipermeable wall. The rotary die process uses two continuous films of capsule lamina-forming material that are brought into convergence between a pair of revolving dies and an injector wedge. The process fills and seals the capsule in dual and coincident operations. In this process, the sheets of capsule lamina-forming material are fed over guide rolls, and then down between the wedge injector and the die rolls. The agent formulation to be encapsulated flows by gravity into a positive displacement pump. The pump meters the active agent formulation through the wedge injector and into the sheets between the die rolls. The bottom of the wedge contains small orifices lined up with the die pockets of the die rolls. The capsule is about half-sealed when the pressure of pumped agent formulation forces the sheets into the die pockets, wherein the capsules are simultaneously filled, shaped, hermetically sealed and cut from the sheets of lamina-forning materials. The sealing of the capsule is achieved by mechanical pressure on the die rolls and by heating of the sheets of lamina-forming materials by the wedge. After manufacture, the agent formulation-filled capsules are dried in the presence of forced air, and a semipermeable lamina encapsulated thereto.

The reciprocating die process produces capsules by leading two films of capsule lamina-forming material between a set of vertical dies. The dies as they close, open, and close perform as a continuous vertical plate forming row after row of pockets across the film. The pockets are filled with agent formulation, and as the pockets move through the dies, they are sealed, shaped, and cut from the moving film as capsules filled with agent formulation. A semipermeable encapsulating lamina is coated thereon to yield the capsule. The continuous process is a manufacturing system that also uses rotary dies, with the added feature that the process can successfully fill active agent in dry powder form into a soft capsule, in addition to encapsulating liquids. The filled capsule of the continuous process is encapsulated with a semipermeable polymeric material to yield the capsule. Procedures for manufacturing soft capsules are disclosed in U.S. Pat. No. 4,627,850 and U.S. Pat. No. 6,419,952.

The dosage forms of the present invention can also be made from an injection-moldable composition by an injection-molding technique. Injection-moldable compositions provided for injection-molding into the semipermeable wall comprise a thermoplastic polymer, or the compositions comprise a mixture of thermoplastic polymers and optional injection-molding ingredients. The thermoplastic polymer that can be used for the present purpose comprise polymers that have a low softening point, for example, below 200° C., preferably within the range of 40° C. to 180° C. The polymers, are preferably synthetic resins, addition polymerized resins, such as polyamides, resins obtained from diepoxides and primary alkanolamines, resins of glycerine and phthalic anhydrides, polymethane, polyvinyl resins, polymer resins with end-positions free or esterified carboxyl or caboxamide groups, for example with acrylic acid, acrylic amide, or acrylic acid esters, polycaprolactone, and its copolymers with dilactide, diglycolide, valerolactone and decalactone, a resin composition comprising polycaprolactone and polyalkylene oxide, and a resin composition comprising polycaprolactone, a polyalkylene oxide such as polyethylene oxide, poly(cellulose) such as poly(hydroxypropylmethylcellulose), poly(hydroxyethylmethylcellulose), and poly(hydroxypropylcellulose). The membrane forming composition can comprise optional membrane-forming ingredients such as polyethylene glycol, talcum, polyvinylalcohol, lactose, or polyvinyl pyrrolidone. The compositions for forming an injection-molding polymer composition can comprise 100% thermoplastic polymer. The composition in another embodiment comprises 10% to 99% of a thermoplastic polymer and 1% to 90% of a different polymer with the total equal to 100%. The invention provides also a thermoplastic polymer composition comprising 1% to 98% of a first thermoplastic polymer, 1% to 90% of a different, second polymer and 1% to 90% of a different, third polymer with all polymers equal to 100%. Representation composition comprises 20% to 90% of thermoplastic polycaprolactone and 10% to 80% of poly(alkylene oxide); a composition comprising 20% to 90% polycaprolactone and 10% to 60% of poly(ethylene oxide) with the ingredients equal to 100%; a composition comprising 10% to 97% of polycaprolactone, 10% to 97% poly(alkylene oxide), and 1% to 97% of poly(ethylene glycol) with all ingredients equal to 100%; a composition comprising 20% to 90% polycaprolactone and 10% to 80% of poly(hydroxypropylcellulose) with all ingredients equal to 100%; and a composition comprising 1% to 90% polycaprolactone, 1% to 90% poly(ethylene oxide), 1% to 90% poly(hydroxypropylcellulose) and 1% to 90% poly(ethylene glycol) with all ingredients equal to 100%. The percent is expressed as weight percent wt %.

In another embodiment of the invention, a composition for injection-molding to provide a membrane can be prepared by blending a composition comprising a polycaprolactone 63 wt %, polyethylene oxide 27 wt %, and polyethylene glycol 10 wt % in a conventional mixing machine, such as a Moriyamam Mixer at 65° C. to 95° C., with the ingredients added to the mixer in the following addition sequence, polycaprolactone, polyethylene oxide and polyethylene glycol. In one example, all the ingredients are mixed for 135 minutes at a rotor speed of 10 to 20 rpm. Next, the blend is fed to a Baker Perkins Kneader™ extruder at 80° C. to 90° C., at a pump speed of 10 rpm and a screw speed of 22 rpm, and then cooled to 10° C. to 12° C., to reach a uniform temperature. Then, the cooled extruded composition is fed to an Albe Pelletizer, converted into pellets at 250° C., and a length of 5 mm. The pellets next are fed into an injection-molding machine, an Arburg Allrounder™ at 200° F. to 350° C. (93° C. to 177° C.), heated to a molten polymeric composition, and the liquid polymer composition forced into a mold cavity at high pressure and speed until the mold is filled and the composition comprising the polymers are solidified into a preselected shape. The parameters for the injection-molding consists of a band temperature through zone 1 to zone 5 of the barrel of 195° F. (91° C.) to 375° F., (191° C.), an injection-molding pressu of 1818 bar, a speed of 55 cm3/s, and a mold temperature of 75° C. The injection-molding compositions and injection-molding procedures are disclosed in U.S. Pat. No. 5,614,578.

Alternatively, the capsule can be made conveniently in two parts, with one part (the “cap”) slipping over and capping the other part (the “body”) as long as the capsule is deformable under the forces exerted by the expandable layer and seals to prevent leakage of the liquid, active agent formulation from between the telescoping portions of the body and cap. The two parts completely surround and capsulate the internal lumen that contains the liquid, active agent formulation, which can contain useful additives. The two parts can be fitted together after the body is filled with a preselected formulation. The assembly can be done by slipping or telescoping the cap section over the body section, and sealing the cap and body, thereby completely surrounding and encapsulating the formulation of active agent.

Soft capsules typically have a wall thickness that is greater than the wall thickness of hard capsules. For example, soft capsules can, for example, have a wall thickness on the order of 10-40 mils, about 20 mils being typical, whereas hard capsules can, for example, have a wall thickness on the order of 2-6 mils, about 4 mils being typical.

In one embodiment of the dosage system, a soft capsule can be of single unit construction and can be surrounded by an unsymmetrical hydro-activated layer as the expandable layer. The expandable layer will generally be unsymmetrical and have a thicker portion remote from the exit orifice. As the hydro-activated layer imbibes and/or absorbs external fluid, it expands and applies a push pressure against the wall of capsule and optional barrier layer and forces active agent formulation through the exit orifice. The presence of an unsymmetrical layer functions to assure that the maximum dose of agent is delivered from the dosage form, as the thicker section of layer distant from passageway swells and moves towards the orifice.

In yet another configuration, the expandable layer can be formed in discrete sections that do not entirely encompass an optionally barrier layer-coated capsule. The expandable layer can be a single element that is formed to fit the shape of the capsule at the area of contact. The expandable layer can be fabricated conveniently by tableting to form the concave surface that is complementary to the external surface of the barrier-coated capsule. Appropriate tooling such as a convex punch in a conventional tableting press can provide the necessary complementary shape for the expandable layer. In this case, the expandable layer is granulated and compressed, rather than formed as a coating. The methods of formation of an expandable layer by tableting are well known, having been described, for example in U.S. Pat. Nos. 4,915,949; 5,126,142; 5,660,861; 5,633,011; 5,190,765; 5,252,338; 5,620,705; 4,931,285; 5,006,346; 5,024,842; and 5,160,743.

In some embodiments, a barrier layer can be first coated onto the capsule and then the tableted, expandable layer is attached to the barrier-coated capsule with a biologically compatible adhesive. Suitable adhesives include, for example, starch paste, aqueous gelatin solution, aqueous gelatin/glycerin solution, acrylate-vinylacetate based adhesives such as Duro-Tak adhesives (National Starch and Chemical Company), aqueous solutions of water soluble hydrophilic polymers such as hydroxypropyl methyl cellulose, hydroxymethyl cellulose, hydroxyethyl cellulose, and the like. That intermediate dosage form can be then coated with a semipermeable layer. The exit orifice is formed in the side or end of the capsule opposite the expandable layer section. As the expandable layer imbibes fluid, it will swell. Since it is constrained by the semipermeable layer, as it expands it will compress the barrier-coated capsule and express the liquid, active agent formulation from the interior of the capsule into the environment of use.

The hard capsules are typically composed of two parts, a cap and a body, which are fitted together after the larger body is filled with a preselected appropriate formulation. This can be done by slipping or telescoping the cap section over the body section, thus completely surrounding and encapsulating the useful agent formulation. Hard capsules can be made, for example, by dipping stainless steel molds into a bath containing a solution of a capsule lamina-forming material to coat the mold with the material. Then, the molds are withdrawn, cooled, and dried in a current of air. The capsule is stripped from the mold and trimmed to yield a lamina member with an internal lumen. The engaging cap that telescopically caps the formulation receiving body is made in a similar manner. Then, the closed and filled capsule can be encapsulated with a semipermeable lamina. The semipermeable lamina can be applied to capsule parts before or after parts and are joined into the final capsule. In another embodiment, the hard capsules can be made with each part having matched locking rings near their opened end that permit joining and locking together the overlapping cap and body after filling with formulation. In this embodiment, a pair of matched locking rings are formed into the cap portion and the body portion, and these rings provide the locking means for securely holding together the capsule. The capsule can be manually filled with the formulation, or they can be machine filled with the formulation. In the final manufacture, the hard capsule is encapsulated with a semipermeable lamina permeable to the passage of fluid and substantially impermeable to the passage of useful agent. Methods of forming hard cap dosage forms are described in U.S. Pat. No. 6,174,547, U.S. Pat. Nos. 6,596,314, 6,419,952, and 6,174,547.

The hard and soft capsules can comprise, for example, gelatin; gelatin having a viscosity of 15 to 30 millipoises and a bloom strength up to 150 grams; gelatin having a bloom value of 160 to 250; a composition comprising gelatin, glycerine, water and titanium dioxide; a composition comprising gelatin, erythrosin, iron oxide and titanium dioxide; a composition comprising gelatin, glycerine, sorbitol, potassium sorbate and titanium dioxide; a composition comprising gelatin, acacia glycerine, and water; and the like. Materials useful for forming capsule wall are known in U.S. Pat. Nos. 4,627,850; and in 4,663,148. Alternatively, the capsules can be made out of materials other than gelatin (see for example, products made by BioProgres plc).

The capsules typically can be provided, for example, in sizes from about 3 to about 22 minims (1 minimim being equal to 0.0616 ml) and in shapes of oval, oblong or others. They can be provided in standard shape and various standard sizes, conventionally designated as (000), (00), (0), (1), (2), (3), (4), and (5). The largest number corresponds to the smallest size. Non-standard shapes can be used as well. In either case of soft capsule or hard capsule, non-conventional shapes and sizes can be provided if required for a particular application.

The osmotic devices of the present invention comprise a semipermeable wall permeable to the passage of exterior biological fluid and substantially impermeable to the passage of drug formulation. The selectively permeable composition used for forming the wall are essentially non-erodible and they are insoluble in biological fluids during the life of the osmotic system. The semipermeable wall comprises a composition that does not adversely affect the host, the drug formulation, an osmopolymer, osmagent and the like. Representative polymers for forming semipermeable wall comprise semipermeable homopolymers, semipermeable copolymers, and the like. In one presently preferred embodiment, the compositions can comprise cellulose esters, cellulose ethers, and cellulose ester-ethers. The cellulosic polymers typically have a degree of substitution, “D.S.”, on their anhydroglucose unit from greater than 0 up to 3 inclusive. By degree of substitution is meant the average number of hydroxyl groups originally present on the anhydroglucose unit that are replaced by a substituting group, or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkylsulfamate, semipermeable polymer forming groups, and the like. The semipermeable compositions typically include a member selected from the group consisting of cellulose acylate, cellulose diacylate, cellulose triacylate, cellulose triacetate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers can include, for example, cellulose acetate have a D.S. of 1.8 to 2.3 and an acetyl content of 32 to 39.9%; cellulose diacetate having a D.S. of 1 to 2 and an acetyl content of 21 to 35%, cellulose triacetate having a D.S. of 2 to 3 and an acetyl content of 34 to 44.8%, and the like. More specific cellulosic polymers include cellulose propionate having a D.S. of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a D.S. of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a D.S. of 2.6 to 3 such as cellulose trivalerate, cellulose trilamate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, cellulose dicarpylate, and the like; mixed cellulose esters such as cellulose acetate valerate, cellulose acetate succinate, cellulose propionate succinate, cellulose acetate octanoate, cellulose valerate palmitate, cellulose acetate heptonate, and the like. Semipermeable polymers are known in U.S. Pat. No. 4,077,407 and they can be synthesized by procedures described in Encyclopedia of Polymer Science and Technology, Vol. 3, pages 325 to 354, 1964, published by Interscience Publishers, Inc., New York. Additional semipermeable polymers for forming the semipermeable wall can comprise, for example, cellulose acetaldehyde dimethyl acetate; cellulose acetate ethylcarbamate; cellulose acetate methylcarbamate; cellulose dimethylaminoacetate; semipermeable polyamide; semipermeable polyurethanes; semipermeable sulfonated polystyrenes; cross-linked selectively semipermeable polymers formed by the coprecipitation of a polyanion and a polycation as disclosed in U.S. Pat. Nos. 3,173,876; 3,276,586; 3,541,005; 3,541,006; and 3,546,142; semipermeable polymers as disclosed in U.S. Pat. No. 3,133,132; semipermeable polystyrene derivatives; semipermeable poly (sodium styrenesulfonate); semipermeable poly (vinylbenzyltremethylammonium chloride); semipermeable polymers, exhibiting a fluid permeability of 10-5 to 10-2 (cc. mil/cm hr.atm) expressed as per atmosphere of hydrostatic or osmotic pressure differences across a semipermeable wall. The polymers are known to the art in U.S. Pat. Nos. 3,845,770; 3,916,899; and 4,160,020; and in Handbook of Common Polymers, by Scott, J. R., and Roff, W. J., 1971, published by CRC Press, Cleveland, Ohio.

The semipermeable wall can also comprise a flux regulating agent. The flux regulating agent is a compound added to assist in regulating the fluid permeability or flux through the wall. The flux regulating agent can be a flux enhancing agent or a decreasing agent. The agent can be preselected to increase or decrease the liquid flux. Agents that produce a marked increase in permeability to fluids such as water are often essentially hydrophilic, while those that produce a marked decrease to fluids such as water are essentially hydrophobic. The amount of regulator in the wall when incorporated therein generally is from about 0.01% to 20% by weight or more. The flux regulator agents in one embodiment that increase flux include, for example, polyhydric alcohols, polyalkylene glycols, polyalkylenediols, polyesters of alkylene glycols, and the like. Typical flux enhancers include polyethylene glycol 300, 400, 600, 1500, 4000, 6000, poly(ethylene glycol-co-propylene glycol), and the like; low molecular weight gylcols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glucol dipropionate, glycerol acetate esters, and the like. Representative flux decreasing agents include, for example, phthalates substituted with an alkyl or alkoxy or with both an alkyl and alkoxy group such as diethyl phthalate, dimethoxyethyl phthalate, dimethyl phthalate, and [di(2-ethylhexyl)phthalate], aryl phthalates such as triphenyl phthalate, and butyl benzyl phthalate; insoluble salts such as calcium sulphate, barium sulphate, calcium phosphate, and the like; insoluble oxides such as titanium oxide; polymers in powder, granule and like form such as polystyrene, polymethylmethacrylate, polycarbonate, and polysulfone; esters such as citric acid esters esterfied with long chain alkyl groups; inert and substantially water impermeable fillers; resins compatible with cellulose based wall forming materials, and the like.

Other materials that can be used to form the semipermeable wall for imparting flexibility and elongation properties to the wall, for making the wall less-to-nonbrittle and to render tear strength, include, for example, phthalate plasticizers such as dibenzyl phthalate, dihexyl phthalate, butyl octyl phthalate, straight chain phthalates of six to eleven carbons, di-isononyl phthalte, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

The semipermeable wall surrounds and forms a compartment containing a plurality of layers, one of which is an expandable layer which in some embodiments, can contain osmotic agents. The expandable layer comprises in one embodiment a hydroactivated composition that swells in the presence of water, such as that present in gastric fluids. Conveniently, it can comprise an osmotic composition comprising an osmotic solute that exhibits an osmotic pressure gradient across the semipermeable layer against an external fluid present in the environment of use. In another embodiment, the hydro-activated layer comprises a hydrogel that imbibes and/or absorbs fluid into the layer through the outer semipermeable wall. The semipermeable wall is non-toxic. It maintains its physical and chemical integrity during operation and it is essentially free of interaction with the expandable layer.

The expandable layer in one preferred embodiment comprises a hydroactive layer comprising a hydrophilic polymer, also known as osmopolymers. The osmopolymers exhibit fluid imbibition properties. The osmopolymers are swellable, hydrophilic polymers, which osmopolymers interact with water and biological aqueous fluids and swell or expand to an equilibrium state. The osmopolymers exhibit the ability to swell in water and biological fluids and retain a significant portion of the imbibed fluid within the polymer structure. The osmopolymers swell or expand to a very high degree, usually exhibiting a 2 to 50 fold volume increase. The osmopolymers can be noncross-linked or cross-linked. The swellable, hydrophilic polymers are in one embodiment lightly cross-linked, such cross-links being formed by covalent or ionic bonds or residue crystalline regions after swelling. The osmopolymers can be of plant, animal or synthetic origin.

The osmopolymers are hydrophilic polymers. Hydrophilic polymers suitable for the present purpose include poly (hydroxy-alkyl methacrylate) having a molecular weight of from 30,000 to 5,000,000; poly (vinylpyrrolidone) having a molecular weight of from 10,000 to 360,000; anionic and cationic hydrogels; polyelectrolytes complexes; poly (vinyl alcohol) having a low acetate residual, cross-linked with glyoxal, formaldehyde, or glutaraldehyde and having a degree of polymerization of from 200 to 30,000; a mixture of methyl cellulose, cross-linked agar and carboxymethyl cellulose; a mixture of hydroxypropyl methylcellulose and sodium carboxymethylcellulose; a mixture of hydroxypropyl ethylcellulose and sodium carboxymethyl cellulose, a mixture of sodium carboxymethylcellulose and methylcellulose, sodium carboxymethylcellulose; potassium carboxymethylcellulose; a water insoluble, water swellable copolymer formed from a dispersion of finely divided copolymer of maleic anhydride with styrene, ethylene, propylene, butylene or isobutylene crosslinked with from 0.001 to about 0.5 moles of saturated cross-linking agent per mole of maleic anhydride per copolymer; water swellable polymers of N-vinyl lactams; polyoxyethylene-polyoxypropylene gel; carob gum; polyacrylic gel; polyester gel; polyuria gel; polyether gel, polyamide gel; polycellulosic gel; polygum gel; initially dry hydrogels that imbibe and absorb water which penetrates the glassy hydrogel and lowers its glass temperature; and the like.

Representative of other osmopolymers can comprise polymers that form hydrogels such as Carbopol™. acidic carboxypolymer, a polymer of acrylic acid cross-linked with a polyallyl sucrose, also known as carboxypolymethylene, and carboxyvinyl polymer having a molecular weight of 250,000 to 4,000,000; Cyanamer™ polyacrylamides; cross-linked water swellable indenemaleic anhydride polymers; Good-rite™ polyacrylic acid having a molecular weight of 80,000 to 200,000; Polyox™ polyethylene oxide polymer having a molecular weight of 100,000 to 5,000,000 and higher; starch graft copolymers; Aqua-Keeps™ acrylate polymer polysaccharides composed of condensed glucose units such as diester cross-linked polygluran; and the like. Representative polymers that form hydrogels are known to the prior art in U.S. Pat. No. 3,865,108; U.S. Pat. No. 4,002,173; U.S. Pat. No. 4,207,893; and in Handbook of Common Polymers, by Scott and Roff, published by the Chemical Rubber Co., Cleveland, Ohio. The amount of osmopolymer comprising a hydro-activated layer can be from about 5% to 100%.

The expandable layer in another manufacture can comprise an osmotically effective compound that comprises inorganic and organic compounds that exhibit an osmotic pressure gradient across a semipermeable wall against an external fluid. The osmotically effective compounds, as with the osmopolymers, imbibe fluid into the osmotic system, thereby making available fluid to push against the inner wall, i.e., in some embodiments, the barrier layer and/or the wall of the soft or hard capsule for pushing active agent from the dosage form. The osmotically effective compounds are known also as osmotically effective solutes, and also as osmagents. Osmotically effective solutes that can be used comprise magnesium sulfate, magnesium chloride, potassium sulfate, sodium sulfate, lithium sulfate, potassium acid phosphate, mannitol, urea, inositol, magnesium succinate, tartaric acid, carbohydrates such as raffinose, sucrose, glucose, lactose, sorbitol, and mixtures therefor. The amount of osmagent in can be from about 5% to 100% of the weight of the layer. The expandable layer optionally comprises an osmopolymer and an osmagent with the total amount of osmopolymer and osmagent equal to 100%. Osmotically effective solutes are known to the prior art as described in U.S. Pat. No. 4,783,337.

In certain embodiments, the dosage forms further can comprise a barrier layer. The barrier layer in certain embodiments is deformable under the pressure exerted by the expandable layer and will be impermeable (or less permeable) to fluids and materials that can be present in the expandable layer, the liquid active agent formulation and in the environment of use, during delivery of the active agent formulation. A certain degree of permeability of the barrier layer can be permitted if the delivery rate of the active agent formulation is not detrimentally affected. However, it is preferred that barrier layer not completely transport through it fluids and materials in the dosage form and the environment of use during the period of delivery of the active agent. The barrier layer can be deformable under forces applied by expandable layer so as to permit compression of capsule to force the liquid, active agent formulation from the exit orifice. In some embodiments, the barrier layer will be deformable to such an extent that it creates a seal between the expandable layer and the semipermeable layer in the area where the exit orifice is formed. In that manner, the barrier layer will deform or flow to a limited extent to seal the initially, exposed areas of the expandable layer and the semipermeable layer when the exit orifice is being formed, such as by drilling or the like, or during the initial stages of operation. When sealed, the only avenue for liquid permeation into the expandable layer is through the semipermeable layer, and there is no back-flow of fluid into the expandable layer through the exit orifice.

Suitable materials for forming the barrier layer can include, for example, polyethylene, polystyrene, ethylene-vinyl acetate copolymers, polycaprolactone and Hytrel™ polyester elastomers (Du Pont), cellulose acetate, cellulose acetate pseudolatex (such as described in U.S. Pat. No. 5,024,842), cellulose acetate propionate, cellulose acetate butyrate, ethyl cellulose, ethyl cellulose pseudolatex (such as Surelease™ as supplied by I0 Colorcon, West Point, Pa. or Aquacoat™ as supplied by FMC Corporation, Philadelphia, Pa.), nitrocellulose, polylactic acid, poly-glycolic acid, polylactide glycolide copolymers, collagen, polyvinyl alcohol, polyvinyl acetate, polyethylene vinylacetate, polyethylene teraphthalate, polybutadiene styrene, polyisobutylene, polyisobutylene isoprene copolymer, polyvinyl chloride, polyvinylidene chloride-vinyl chloride copolymer, copolymers of acrylic acid and methacrylic acid esters, copolymers of methylmethacrylate and ethylacrylate, latex of acrylate esters (such as Eudragit™ supplied by RohmPharma, Darmstaat, Germany), polypropylene, copolymers of propylene oxide and ethylene oxide, propylene oxide ethylene oxide block copolymers, ethylenevinyl alcohol copolymer, polysulfone, ethylene vinylalcohol copolymer, polyxylylenes, polyalkoxysilanes, polydimethyl siloxane, polyethylene glycol-silicone elastomers, electromagnetic irradiation crosslinked acrylics, silicones, or polyesters, thermally crosslinked acrylics, silicones, or polyesters, butadiene-styrene rubber, and blends of the above.

Preferred materials can include cellulose acetate, copolymers of acrylic acid and methacrylic acid esters, copolymers of methylmethacrylate and ethylacrylate, and latex of acrylate esters. Preferred copolymers can include poly (butyl methacrylate), (2-dimethylaminoethyl)methacrylate, methyl methacrylate) 1:2:1, 150,000, sold under the trademark EUDRAGIT E; poly (ethyl acrylate, methyl methacrylate) 2:1, 800,000, sold under the trademark EUDRAGIT NE 30 D; poly (methacrylic acid, methyl methacrylate) 1:1, 135,000, sold under the trademark EUDRAGIT L; poly (methacrylic acid, ethyl acrylate) 1:1, 250,000, sold under the trademark EUDRAGIT L; poly (methacrylic acid, methyl methacrylate) 1:2, 135,000, sold under the trademark EUDRAGIT S; poly (ethyl acrylate, methyl methacrylate, trimethylammonioethyl methacrylate chloride) 1:2:0.2, 150,000, sold under the trademark EUDRAGIT RL; poly (ethyl acrylate, methyl methacrylate, trimethylammonioethyl methacrylate chloride) 1:2:0.1, 150,000, sold as EUDRAGIT RS. In each case, the ratio x:y:z indicates the molar proportions of the monomer units and the last number is the number average molecular weight of the polymer. Especially preferred are cellulose acetate containing plasticizers such as acetyl tributyl citrate and ethylacrylate methylmethylacrylate copolymers such as Eudragit NE.

The foregoing materials for use as the barrier layer can be formulated with plasticizers to make the barrier layer suitably deformable such that the force exerted by the expandable layer will collapse the compartment formed by the barrier layer to dispense the liquid, active agent formulation. Examples of typical plasticizers are as follows: polyhydric alcohols, triacetin, polyethylene glycol, glycerol, propylene glycol, acetate esters, glycerol triacetate, triethyl citrate, acetyl triethyl citrate, glycerides, acetylated monoglycerides, oils, mineral oil, castor oil and the like. The plasticizers can be blended into the material in amounts of 10-50 weight percent based on the weight of the material.

The various layers forming the barrier layer, expandable layer and semipermeable layer can be applied by conventional coating methods such as described in U.S. Pat. No. 5,324,280. While the barrier layer, expandable layer and semipermeable wall have been illustrated and described for convenience as single layers, each of those layers can be composites of several layers. For example, for particular applications it may be desirable to coat the capsule with a first layer of material that facilitates coating of a second layer having the permeability characteristics of the barrier layer. In that instance, the first and second layers comprise the barrier layer. Similar considerations would apply to the semipermeable layer and the expandable layer.

The exit orifice can be formed by mechanical drilling, laser drilling, eroding an erodible element, extracting, dissolving, bursting, or leaching a passageway former from the composite wall. The exit orifice can be a pore formed by leaching sorbitol, lactose or the like from a wall or layer as disclosed in U.S. Pat. No. 4,200,098. This patent discloses pores of controlled-size porosity formed by dissolving, extracting, or leaching a material from a wall, such as sorbitol from cellulose acetate. A preferred form of laser drilling is the use of a pulsed laser that incrementally removes material from the composite wall to the desired depth to form the exit orifice.

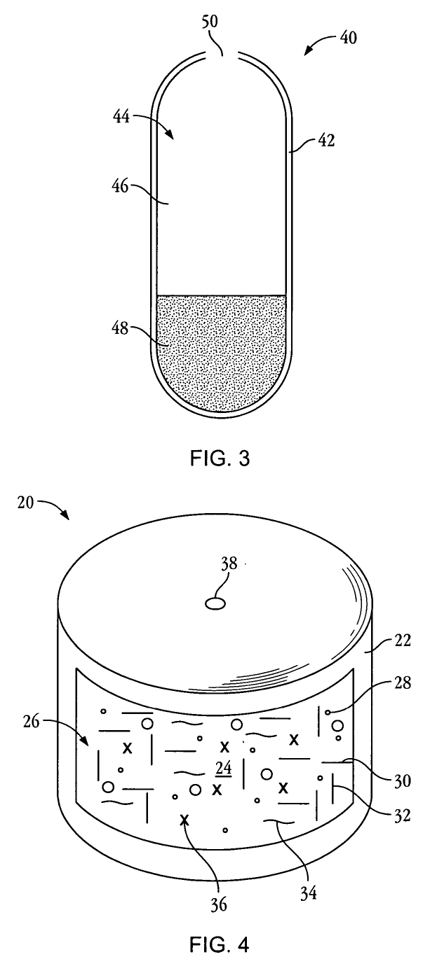

FIG. 3 is a schematic illustration of another exemplary osmotic dosage form. Dosage forms of this type are described in detail in U.S. Pat. Nos.: 4,612,008; 5,082,668; and 5,091,190. In brief, dosage form 40, shown in cross-section, has a semi-permeable wall 42 defining an internal compartment 44. Internal compartment 44 contains a bilayered-compressed core having a drug layer 46 and a push layer 48. As will be described below, push layer 48 is a displacement composition that is positioned within the dosage form such that as the push layer expands during use, the materials forming the drug layer are expelled from the dosage form via one or more exit ports, such as exit port 50. The push layer can be positioned in contacting layered arrangement with the drug layer, as illustrated in FIG. 4, or can have one or more intervening layers separating the push layer and drug layer.

Drug layer 46 comprises tramadol and substances comprising gabapentin in an admixture with pharmaceutical excipients. An exemplary dosage form can have a drug layer comprised of tramadol, a gabapentin, a poly(ethylene oxide) as a carrier, sodium chloride as an osmagent, hydroxypropylmethylcellulose as a binder, and magnesium stearate as a lubricant.

Push layer 48 comprises osmotically active component(s), such as one or more polymers that imbibes an aqueous or biological fluid and swells, referred to in the art as an osmopolymer. Osmopolymers are swellable, hydrophilic polymers that interact with water and aqueous biological fluids and swell or expand to a high degree, typically exhibiting a 2-50 fold volume increase. The osmopolymer can be non-crosslinked or crosslinked, and in a preferred embodiment the osmopolymer is at least lightly crosslinked to create a polymer network that is too large and entangled to easily exit the dosage form during use. Examples of polymers that may be used as osmopolymers are provided in the references noted above that describe osmotic dosage forms in detail. A typical osmopolymer is a poly(alkylene oxide), such as poly(ethylene oxide), and a poly(alkali carboxymethylcellulose), where the alkali is sodium, potassium, or lithium. Additional excipients such as a binder, a lubricant, an antioxidant, and a colorant may also be included in the push layer. In use, as fluid is imbibed across the semi-permeable wall, the osmopolymer(s) swell and push against the drug layer to cause release of the drug from the dosage form via the exit port(s).

The push layer can also include a component referred to as a binder, which is typically a cellulose or vinyl polymer, such as poly-n-vinylamide, poly-n-vinylacetamide, poly(vinyl pyrrolidone), poly-n-vinylcaprolactone, poly-n-vinyl-5-methyl-2-pyrrolidone, and the like. The push layer can also include a lubricant, such as sodium stearate or magnesium stearate, and an antioxidant to inhibit the oxidation of ingredients. Representative antioxidants include, but are not limited to, ascorbic acid, ascorbyl palmitate, butylated hydroxyanisole, a mixture of 2 and 3 tertiary-butyl-4-hydroxyanisole, and butylated hydroxytoluene.

An osmagent may also be incorporated into the drug layer and/or the push layer of the osmotic dosage form. Presence of the osmagent establishes an osmotic activity gradient across the semi-permeable wall. Exemplary osmagents include salts, such as sodium chloride, potassium chloride, lithium chloride, etc. and sugars, such as raffinose, sucrose, glucose, lactose, and carbohydrates.

With continuing reference to FIG. 4, the dosage form can optionally include an overcoat (not shown) for color coding the dosage forms according to dose or for providing an immediate release of tramadol and/or substances comprising gabapentin or other drugs.

In use, water flows across the wall and into the push layer and the drug layer. The push layer imbibes fluid and begins to swell and, consequently, pushes on drug layer 44 causing the material in the layer to be expelled through the exit orifice and into the gastrointestinal tract. Push layer 48 is designed to imbibe fluid and continue swelling, thus continually expelling tramadol and substances comprising gabapentin from the drug layer throughout the period during which the dosage form is in the gastrointestinal tract. In this way, the dosage form provides a supply of tramadol and substances comprising gabapentin to the gastrointestinal tract for a specified window.

In an embodiment, inventive dosage forms comprise two or more forms of tramadol and/or substances comprising gabapentin so that a first form of tramadol and/or substances comprising gabapentin is available for absorption in the upper G.I. tract and a second form is presented for absorption in the lower G.I. tract. This can facilitate optimal absorption in circumstances wherein different characteristics are needed to optimize absorption throughout the G.I. tract. Such an embodiment may be preferably achievable using a tri-layered oral osmotic dosage form

An exemplary dosage form, referred to in the art as an elementary osmotic pump dosage form, is shown in FIG. 4. Dosage form 20, shown in a cutaway view, is also referred to as an elementary osmotic pump, and is comprised of a semi-permeable wall 22 that surrounds and encloses an internal compartment 24. The internal compartment contains a single component layer referred to herein as a drug layer 26, comprising tramadol and substances comprising gabapentin 28 in an admixture with selected excipients. The excipients are adapted to provide an osmotic activity gradient for attracting fluid from an external environment through wall 22 and for forming deliverable tramadol and substances comprising gabapentin formulation upon imbibition of fluid. The excipients may include a suitable suspending agent, also referred to herein as drug carrier 30, a binder 32, a lubricant 34, and an osmotically active agent referred to as an osmagent 36. Exemplary materials for each of these components are provided below.

Semi-permeable wall 22 of the osmotic dosage form is permeable to the passage of an external fluid, such as water and biological fluids, but is substantially impermeable to the passage of components in the internal compartment. Materials useful for forming the wall are essentially nonerodible and are substantially insoluble in biological fluids during the life of the dosage form. Representative polymers for forming the semi-permeable wall have been discussed elsewehere herein, and include homopolymers and copolymers, such as, cellulose esters, cellulose ethers, and cellulose ester-ethers. Flux-regulating agents can be admixed with the wall-forming material to modulate the fluid permeability of the wall, as discussed elsewhere herein. For example, agents that produce a marked increase in permeability to fluid such as water are often essentially hydrophilic, while those that produce a marked permeability decrease to water are essentially hydrophobic. Exemplary flux regulating agents include those discussed elsewhere herein, together with polyhydric alcohols, polyalkylene glycols, polyalkylenediols, polyesters of alkylene glycols, and the like.

In operation, the osmotic gradient across wall 22 due to the presence of osmotically-active agents causes gastric fluid to be imbibed through the wall, swelling of the drug layer, and formation of a deliverable formulation of tramadol and substances comprising gabapentin (e.g., a solution, suspension, slurry or other flowable composition) within the internal compartment. The deliverable formulation is released through an exit 38 as fluid continues to enter the internal compartment. Even as drug formulation is released from the dosage form, fluid continues to be drawn into the internal compartment, thereby driving continued release. In this manner, tramadol and substances comprising gabapentin are released in a sustained manner over an extended time period.

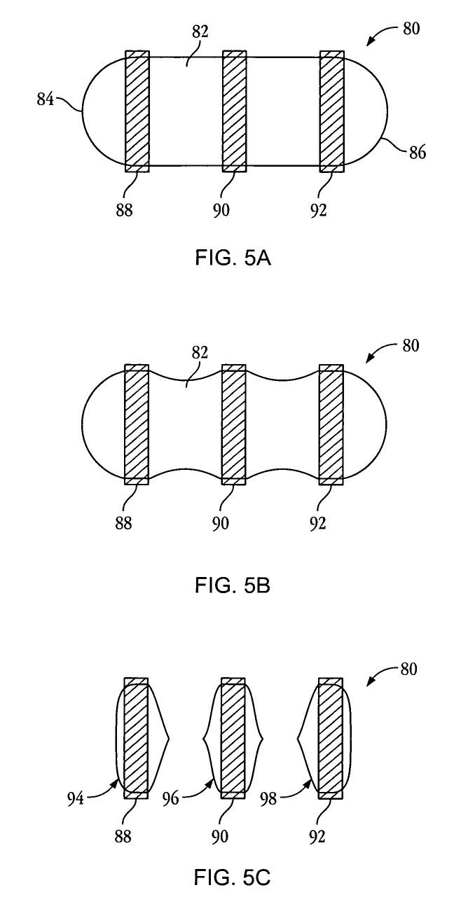

FIGS. 5A-5C illustrate another exemplary dosage form, known in the art and described in U.S. Pat. Nos. 5,534,263; 5,667,804; and 6,020,000. Briefly, a cross-sectional view of a dosage form 80 is shown prior to ingestion into the gastrointestinal tract in FIG. 5A. The dosage form is comprised of a cylindrically shaped matrix 82 comprising tramadol and substances comprising gabapentin. Ends 84, 86 of matrix 82 are preferably rounded and convex in shape in order to ensure ease of ingestion. Bands 88, 90, and 92 concentrically surround the cylindrical matrix and are formed of a material that is relatively insoluble in an aqueous environment. Suitable materials are set forth in the patents noted above.

After ingestion of dosage form 80, regions of matrix 82 between bands 88, 90, 92 begin to erode, as illustrated in FIG. 5B. Erosion of the matrix initiates release of tramadol and substances comprising gabapentin into the fluidic environment of the G.I. tract. As the dosage form continues transit through the G.I. tract, the matrix continues to erode, as illustrated in FIG. 5C. Here, erosion of the matrix has progressed to such an extent that the dosage form breaks into three pieces, 94, 96, 98. Erosion will continue until the matrix portions of each of the pieces have completely eroded. Bands 94, 96, 98 will thereafter be expelled from the G.I. tract.

In an embodiment, the inventive controlled delivery dosage forms comprise gastric retention dosage forms. U.S. Pat. No. 5,007,790 to Shell, granted Apr. 16, 1991 and entitled Sustained-release oral drug dosage form (“Shell”) discloses a gastric retention dosage form useful in the practice of this invention. Shell discloses sustained-release oral drug-dosage forms that release drug in solution at a rate controlled by the solubility of the drug. The dosage form comprises a tablet or capsule which comprises a plurality of particles of a dispersion of a limited solubility drug in a hydrophilic, water-swellable, crosslinked polymer that maintains its physical integrity over the dosing lifetime but thereafter rapidly dissolves. Once ingested, the particles swell to promote gastric retention and permit the gastric fluid to penetrate the particles, dissolve drug and leach it from the particles. Tramadol and substances that comprise gabapentin may be incorporated into such a gastric retention dosage form, or others known in the art, in the practice of this invention.

It will be appreciated the dosage forms described in FIGS. 1-5 are merely exemplary of a variety of dosage forms designed for and capable of achieving delivery of the inventive moiety complex to the G.I. tract. Those of skill in the pharmaceutical arts can identify other dosage forms that would be suitable.

Typical doses of tramadol and substances that comprise gabapentin in the inventive dosage forms may vary broadly. The inventors note that the molecular weight of substances that comprise gabapentin may vary significantly depending on whether it is administered as a loose ion-pair salt, a complex, a structural homolog, and so on. Therefore, the dosage strength of substances that comprise gabapentin may need to be varied as the form incorporated into the dosage form is varied. The dose administered is generally adjusted in accord with the desired result for individual patients.

In an aspect, the invention provides a method for treating an indication, such as a disease or disorder, preferably a disease or disorder amenable to treatment by administration of tramadol and substances that comprise gabapentin, in a patient by administering a controlled delivery dosage form that comprises tramadol and substances that comprise gabapentin. In one embodiment, a composition comprising tramadol and substances that comprise gabapentin, and a pharmaceutically-acceptable vehicle, is administered to the patient via oral administration.

The present invention is further directed to a method of treatment comprising administering to a patient in need thereof, an oral controlled delivery dosage form comprising tramadol and substances that comprise gabapentin wherein the tramadol and substances that comprise gabapentin are released from the dosage form at a substantially zero order rate of release, preferably a zero order rate of release. A variety of controlled delivery dosage forms disclosed herein are capable of providing a substantially zero order rate of release, preferably a zero order rate of release. Such dosage forms comprise elementary osmotic pumps, matrix, and bi-layered osmotic dosage forms, as well as others known to one of skill in the art.

The ascending release rate embodiments are particularly useful in circumstances wherein the lower G.I. absorption is still less than the upper G.I. absorption. In such case, the ascending release rate can compensate in part for reduced lower G.I. absorption or even reduced absorption in areas of the upper G.I. that do not posses high levels of the active transporters that may be responsible for the primary transport of gabapentin. In one ascending rate of release embodiment, the release rate over the first approximately 3 hours after dosing of an inventive dosage form is approximately I/F fold that of the release rate beyond approximately 3 hours after dosing where
F=X/Y
and wherein X=bioavailability of gabapentin when delivered to the lower G.I., and Y=bioavailability of gabapentin when delivered to the upper G.I. Various ascending release rate profiles can be obtained by one of skill in the art by optimizing appropriate formulations. For instance, of skill in the art could adjust the dosage form shown in FIG. 5 to varying release rates so as to achieve a desired ascending release rate profile. Such adjustments are known to one of skill in the art.

In one embodiment, the inventive dosage forms may achieve an ascending release rate through the provision of more than one drug layer. In osmotic devices with multiple drug layers, a drug concentration gradient between the layers facilitates the achievement of an ascending drug release rate for an extended time period. For example, in one embodiment of the present invention, the osmotic dosage form comprises a first drug layer and a second drug layer, wherein the concentration of substances comprising gabapentin contained within the first drug layer is greater than the concentration of substances comprising gabapentin contained within the second drug layer, and the expandable layer is contained within a third layer. In outward order from the core of the dosage form is the expandable layer, the second drug layer, and the first drug layer. In operation through the cooperation of the dosage form components, substances comprising gabapentin are successively released, in a sustained and controlled manner, from the second drug layer and then from the first drug layer such that an ascending release rate over an extended time period is achieved.

The present invention is further directed to pharmaceutical compositions, as that term is defmed herein, and to methods of administering pharmaceutical compositions to a patient in need thereof. Preferably the present invention is directed to methods of administering pharmaceutical compositions to a patient in need thereof in therapeutically effective amounts.

In an embodiment, the invention relates to an oral dosage form comprising: an oral controlled delivery dosing structure comprising structure that controllably delivers tramadol and a substance that comprises gabapentin; and wherein the weight ratio of gabapentin equivalent to tramadol equivalent present in the oral dosage form ranges from about 0.75:1 to about 6.5:1; and wherein the total weight of the tramadol and substance that comprises gabapentin present in the oral dosage form is less than about 1500 milligrams; and wherein the oral controlled delivery dosing structure is adapted to controllably deliver the substance that comprises gabapentin at a rate that is effective to, after a single administration of the oral dosage form to a patient, maintain a gabapentin plasma drug concentration that is at least about twenty-five percent of a gabapentin Cmax throughout a window of at least about fifteen hours duration after a time at which the gabapentin Cmax occurs. Preferably, the tramadol comprises tramadol HCl; the substance that comprises gabapentin comprises a complex that comprises gabapentin and an alkyl sulfate salt; the window is of at least about eighteen hours duration after the time at which the gabapentin Cmax occurs; the weight ratio of gabapentin equivalent to tramadol equivalent present in the oral dosage form ranges from about 0.80:1 to about 5.5:1; or the oral dosage form comprises an osmotic oral dosage form.

In an embodiment, the invention relates to an oral dosage form comprising: an oral controlled delivery dosing structure comprising structure that controllably delivers tramadol and a substance that comprises gabapentin; wherein the weight ratio of gabapentin equivalent to tramadol equivalent present in the oral dosage form ranges from about 0.75:1 to about 6.5:1; and wherein the total weight of the tramadol and substance that comprises gabapentin present in the oral dosage form is less than about 1500 milligrams; and wherein the controlled delivery dosing structure is adapted to controllably deliver the substance that comprises gabapentin contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs, wherein the wt % is based on the total weight of the substance that comprises gabapentin present in the controlled delivery dosage form. Preferably, the tramadol comprises tramadol HCl; the substance that comprises gabapentin comprises a complex that comprises gabapentin and an alkyl sulfate salt; the weight ratio of gabapentin equivalent to tramadol equivalent present in the oral dosage form ranges from about 0.80:1 to about 5.5:1; or the oral dosage form comprises an osmotic oral dosage form.

In another embodiment, the invention relates to an oral dosage form comprising: an oral controlled delivery dosing structure comprising structure that controllably delivers tramadol and a substance that comprises gabapentin; wherein the weight ratio of gabapentin equivalent to tramadol equivalent present in the oral dosage form ranges from about 0.75:1 to about 6.5:1; and wherein the total weight of the tramadol and substance that comprises gabapentin present in the oral dosage form is less than about 1500 milligrams; and wherein the controlled delivery dosing structure is adapted to controllably deliver the portion of the substance that comprises tramadol contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs, wherein the wt % is based on the total weight of the tramadol present in the controlled delivery dosage form. Preferably, the tramadol comprises tramadol HCl; the substance that comprises gabapentin comprises a complex that comprises gabapentin and an alkyl sulfate salt; the weight ratio of gabapentin equivalent to tramadol equivalent present in the oral dosage form ranges from about 0.80:1 to about 5.5:1; or the oral dosage form comprises an osmotic oral dosage form.

In still another embodiment, the invention relates to an oral controlled delivery dosage form comprising: an oral controlled delivery dosing structure comprising structure that controllably delivers (i) a substance that comprises gabapentin, and (ii) tramadol; wherein the oral controlled delivery dosing structure is adapted to controllably deliver the substance that comprises gabapentin at a release rate that satisfies the following relationship: Rate₀₋₃=(1/F) * Rate₃₋₁₀ wherein Rate₀₋₃ represents a mean release rate for about a three hour period immediately following administration of the dosage form, Rate₃₋₁₀ represents a mean release rate for a period from about three hours immediately following administration of the oral dosage form to about ten hours immediately following administration of the oral dosage form, and F=X/Y, wherein X=a colonic bioavailability of gabapentin and Y=upper gastrointestinal tract bioavailability of gabapentin.

Preferably, the tramadol comprises tramadol HCl; or the substance that comprises gabapentin comprises a complex that comprises gabapentin and an alkyl sulfate salt.

In an embodiment, the invention relates to a method comprising (1) providing an oral dosage form comprising: an oral controlled delivery dosing structure comprising structure that controllably delivers tramadol and a substance that comprises gabapentin; and wherein the weight ratio of gabapentin equivalent to tramadol equivalent present in the oral dosage form ranges from about 0.75:1 to about 6.5:1; and wherein the total weight of the tramadol and substance that comprises gabapentin present in the oral dosage form is less than about 1500 milligrams; and wherein the oral controlled delivery dosing structure is adapted to controllably deliver the substance that comprises gabapentin at a rate that is effective to, after a single administration of the oral dosage form to a patient, maintain a gabapentin plasma drug concentration that is at least about twenty-five percent of a gabapentin Cmax throughout a window of at least about fifteen hours duration after a time at which the gabapentin Cmax occurs; and (2) administering the oral dosage form to a patient. Preferably, the tramadol comprises tramadol HCl; the substance that comprises gabapentin comprises a complex that comprises gabapentin and an alkyl sulfate salt; the window is of at least about eighteen hours duration after the time at which the gabapentin Cmax occurs; the weight ratio of gabapentin equivalent to tramadol equivalent present in the oral dosage form ranges from about 0.80:1 to about 5.5:1; or the oral dosage form comprises an osmotic oral dosage form.

In yet another embodiment, the invention relates to a method comprising: (1) providing an oral dosage form comprising: an oral controlled delivery dosing structure comprising structure that controllably delivers tramadol and a substance that comprises gabapentin; wherein the weight ratio of gabapentin equivalent to tramadol equivalent present in the oral dosage form ranges from about 0.75:1 to about 6.5:1; and wherein the total weight of the tramadol and substance that comprises gabapentin present in the oral dosage form is less than about 1500 milligrams; and wherein the controlled delivery dosing structure is adapted to controllably deliver the substance that comprises gabapentin contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs, wherein the wt % is based on the total weight of the substance that comprises gabapentin present in the controlled delivery dosage form; and (2) administering the oral dosage form to a patient. Preferably, the tramadol comprises tramadol HCl; the substance that comprises gabapentin comprises a complex that comprises gabapentin and an alkyl sulfate salt; the weight ratio of gabapentin equivalent to tramadol equivalent present in the oral dosage form ranges from about 0.80:1 to about 5.5:1; or the oral dosage form comprises an osmotic oral dosage form.

In an embodiment, the invention relates to a method comprising: (1) providing an oral dosage form comprising: an oral controlled delivery dosing structure comprising structure that controllably delivers tramadol and a substance that comprises gabapentin; wherein the weight ratio of gabapentin equivalent to tramadol equivalent present in the oral dosage form ranges from about 0.75:1 to about 6.5:1; and wherein the total weight of the tramadol and substance that comprises gabapentin present in the oral dosage form is less than about 1500 milligrams; and wherein the controlled delivery dosing structure is adapted to controllably deliver the portion of the substance that comprises tramadol contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs, wherein the wt % is based on the total weight of the tramadol present in the controlled delivery dosage form; and (2) administering the oral dosage form to a patient. Preferably, the tramadol comprises tramadol HCl; the substance that comprises gabapentin comprises a complex that comprises gabapentin and an alkyl sulfate salt; the weight ratio of gabapentin equivalent to tramadol equivalent present in the oral dosage form ranges from about 0.80:1 to about 5.5:1; or the oral dosage form comprises an osmotic oral dosage form.

In a further embodiment, the invention relates to a method comprising: (1) providing an oral controlled delivery dosage form comprising an oral controlled delivery dosing structure comprising structure that controllably delivers: (i) a substance that comprises gabapentin, and (ii) tramadol; wherein the oral controlled delivery dosing structure is adapted to controllably deliver the substance that comprises gabapentin at a release rate that satisfies the following relationship: Rateo₀₋₃=(1/F) * Rate₃₋₁₀ wherein Rate₀₋₃ represents a mean release rate for about a three hour period immediately following administration of the dosage form, Rate₃₋₁₀ represents a mean release rate for a period from about three hours immediately following administration of the oral dosage form to about ten hours immediately following administration of the oral dosage form, and F=X/Y, wherein X=a colonic bioavai lability of gabapentin and Y=upper gastrointestinal tract bioavailability of gabapentin; and (2) administering the dosage form to a patient. Preferably, the tramadol comprises tramadol HCl; or the substance that comprises gabapentin comprises a complex that comprises gabapentin and an alkyl sulfate salt.

In still a further embodiment, the invention relates to a pharmaceutical composition comprising a substance comprising a complex that comprises (i) gabapentin and (ii) a transport moiety; and tramadol. Preferably, the transport moiety comprises an alkyl sulfate salt; the alkyl sulfate salt comprises sodium lauryl sulfate; or the substance excludes substances that comprise gabapentin prodrugs wherein the gabapentin prodrug comprises chemical structure that enhances colonic absorption of the gabapentin prodrug as compared to gabapentin. In another preferred embodiment, there is provided an oral dosage form comprising the pharmaceutical composition; the oral dosage form comprises an oral controlled delivery dosage form; the oral dosage form comprises an osmotic oral controlled delivery dosage form; the osmotic oral controlled delivery dosage form comprises a solid osmotic oral controlled delivery dosage form; or the osmotic oral controlled delivery dosage form comprises a liquid osmotic oral controlled delivery dosage form.

In yet another embodiment, the invention relates to a method comprising: (1) providing a pharmaceutical composition comprising a substance comprising a complex that comprises (i) gabapentin and (ii) a transport moiety; and tramadol; and (2) administering the pharmaceutical composition to a patient. Preferably, the transport moiety comprises an alkyl sulfate salt; the alkyl sulfate salt comprises sodium lauryl sulfate; or the substance excludes substances that comprise gabapentin prodrugs wherein the gabapentin prodrug comprises chemical structure that enhances colonic absorption of the gabapentin prodrug as compared to gabapentin. In another preferred embodiment, there are provided methods wherein the oral dosage form disclosed above is provided and administered to a patient; wherein the oral dosage form comprises an oral controlled delivery dosage form; wherein the oral dosage form comprises an osmotic oral controlled delivery dosage form; wherein the osmotic oral controlled delivery dosage form comprises a solid osmotic oral controlled delivery dosage form; or wherein the osmotic oral controlled delivery dosage form comprises a liquid osmotic oral controlled delivery dosage form.

Although the foregoing invention has been described in detail by way of example for purposes of clarity of understanding, it will be apparent to persons skilled in the art that certain changes and modifications are comprehended by the disclosure and can be practiced without undue experimentation within the scope of the appended claims, which are presented by way of illustration not limitation.

The inventive compositions are generally formulated as sterile, substantially isotonic and in full compliance with all Good Manufacturing Practice (GMP) regulations of the U.S. Food and Drug Administration. Depending on the dose of drug desired to be administered, one or more of the oral dosage forms can be administered.

All publications and patent documents cited above are hereby incorporated by reference in their entirety for all purposes to the same extent as if each were so individually denoted.

Each recited range includes all combinations and subcombinations of ranges, as well as specific numerals contained therein.

### IV. EXAMPLES

The following examples are illustrative of the present invention and should not be considered as limiting the scope of the invention in any way, as these examples and other equivalents thereof will become apparent to those versed in the art in light of the present disclosure, drawings and accompanying claims.

### Example 1

Preparation of Gabapentin—Lauryl Sulfate Complex

1. A solution of 0.5 mL 36.5% hydrochloric acid (5 mmol HCl) in 25 mL deionized water was prepared.

2. 5 mmol gabapentin (0.86 g) was added to the solution in step 1. The mixture was stirred for 10 min at room temperature. Gabapentin hydrochloride was formed.

3. 5 mmol sodium lauryl sulfate (1.4 g) was added to the aqueous solution in step 2. The mixture was stirred for 20 min at room temperature.

4. 50 mL dichloromethane was added to the solution in step 3. The mixture was stirred for 2 hours at room temperature.

5. The mixture of step 4 was transferred to a separatory funnel and allowed to settle for 3 hours. Two phases were formed, a lower phase of dichloromethane and an upper phase of water.

6. The upper and lower phases in step 5 were separated. The lower dichloromethane phase was recovered and the dichloromethane was evaporated to dryness at room temperature, followed by drying in a vacuum oven for 4 hours at 40° C. A complex of gabapentin-lauryl sulfate (1.9 g) was obtained. Total yield was 87% relative to theoretical amount calculated from the initial amounts of gabapentin and sodium lauryl sulfate.

### Example 2

Preparation of Gabapentin—Decyl Sulfate Complex

1. A solution of 0.5 mL 36.5% hydrochloric acid (5 mmol HCl) in 25 mL deionized water was prepared.

2. 5 mmol gabapentin (0.86 g) was added to the solution in step 1. The mixture was stirred for 10 min at room temperature. Gabapentin hydrochloride was formed.

3. 5 mmol sodium decyl sulfate (1.3 g) was added to the aqueous solution in step 2. The mixture was stirred for 10 min at room temperature.

4. 50 mL dichloromethane was added to the solution in step 3. The mixture was stirred for 2 hours at room temperature.

5. The mixture of step 4 was transferred to a separatory funnel and allowed to settle for 3 hours. Two phases were formed, a lower phase of dichloromethane and an upper phase of water.

6. The upper and lower phases in step 5 were separated. The lower dichloromethane phase was recovered and the dichloromethane was evaporated to dryness at room temperature, followed by drying in a vacuum oven for 4 hours at 40° C. A past-like complex of gabapentin-decyl sulfate (1.91 g) was obtained. Total yield was 93% relative to theoretical amount calculated from the initial amounts of gabapentin and sodium decyl sulfate.

### Example 3

Solid Osmotic Dosage Form for Delivery of Gabapentin—Lauryl Sulfate Complex and Tramadol HCl

A dosage form is prepared as follows: (100 mg tramadol HCl/511 mg gabapentin lauryl sulfate, i.e. 200 mg gabapentin equivalent)

The gabapentin—lauryl sulfate complex and tramadol HCl layer in the dosage form is prepared as follows. First, 7.78 grams of gabapentin-lauryl sulfate complex, prepared as described in Example 1, 1.52 grams of tramadol HCl, 0.50 g polyethylene oxide of 5,000,000 molecular weight, 0.10 g of polyvinylpyrrolidone having molecular weight of about 38,000 are dry blended in a conventional blender for 20 minutes to yield a homogenous blend. Next, denatured anhydrous ethanol is added slowly to the blend with continuous mixing for 5 minutes. The blended wet composition is passed through a 16 mesh screen and dried overnight at room temperature. Then, the dry granules are passed through a 16 mesh screen and 0.10 g magnesium stearate are added and all the dry ingredients are dry blended for 5 minutes. The composition is comprised of 77.8 wt % gabapentin—lauryl sulfate complex, 15.2 wt % tramadol HCl, 5.0 wt % polyethylene oxide 5,000,000 molecular weight, 1.0 wt % polyvinylpyrrolidone having molecular weight of about 35,000 to 40,000 and 1.0 wt % magnesium stearate.

A push layer comprised of an osmopolymer hydrogel composition is prepared as follows. First, 637.70 g of pharmaceutically acceptable polyethylene oxide comprising a 7,000,000 molecular weight, 300 g sodium chloride and 10 g ferric oxide are separately screened through a 40 mesh screen. The screened ingredients are mixed with 50 g of hydroxypropylmethylcellulose of 9,200 molecular weight to produce a homogenous blend. Next, 150 mL of denatured anhydrous alcohol is added slowly to the blend with continuous mixing for 5 minutes. Then, 0.80 g of butylated hydroxytoluene is added followed by more blending. The freshly prepared granulation is passed through a 20 mesh screen and allowed to dry for 20 hours at room temperature (ambient). The dried ingredients are passed through a 20 mesh screen and 2.50 g of magnesium stearate is added and all the ingredients are blended for 5 minutes. The final composition is comprised of 63.67 wt % of polyethylene oxide, 30.00 wt % sodium chloride, 1.00 wt % ferric oxide, 5.00 wt % hydroxypropylmethylcellulose, 0.08 wt % butylated hydroxytoluene and 0.25 wt % magnesium stearate.

The bi-layer dosage form is prepared as follows. First, 657 mg of the drug layer composition is added to a punch and die set and tamped. Then, 328 mg of the hydrogel composition is added and the two layers compressed under a compression force of 1.0 ton (1000 kg) into a 9/32 inch (0.714 cm) diameter punch die set, forming an intimate bi-layered core (tablet).

A semipermeable wall-forming composition is prepared comprising 80.0 wt % cellulose acetate having a 39.8% acetyl content and 20.0% polyoxyethylene-polyoxypropylene copolymer having a molecular weight of 7680-9510 by dissolving the ingredients in acetone in a 80:20 wt/wt composition to make a 5.0% solids solution. Placing the solution container in a warm water bath during this step accelerates the dissolution of the components. The wall-forming composition is sprayed onto and around the bi-layered core to provide a 60 to 80 mg thickness semi-permeable wall.

Next, a 40 mil (1.02 mm) exit orifice is laser drilled in the semipermeable walled bi-layered tablet to provide contact of the drug containing layer with the exterior of the delivery device. The dosage form is dried to remove any residual solvent and water.

The release rate for the dosage form made according to Example 5 is tested on the Distek 5100 (USP apparatus 2 paddle tester) in 900 mL artificial intestinal fluid (AIF, pH=6.8). The temperature of the dissolution medium was maintained at 37° C. and the paddle speed was 100 rpm. The concentration of gabapentin and of tramadol HCl is measured with HPLC. Two systems are tested.

### Example 4

Liquid Osmotic Dosage Form Comprising a Gabapentin Complex and Tramadol HCl (50 mg tramadol HCl/120 mg gabapentin decyl sulfate, i.e. 50 mg gabapentin equivalent)

A hard cap oral osmotic device system for dispensing the complex of Example 2 and tramadol HCl in the G.I. tract may be prepared as follows:

First, an osmotic push-layer formation is granulated using a Glatt fluid bed granulator (FBG). The composition of the push granules is comprised of 63.67 wt % of polyethylene oxide of 7,000,000 molecular weight, 30.00 wt % sodium chloride, 1.00 wt % ferric oxide, 5.00 wt % hydroxypropylmethylcellulose of 9,200 molecular weight, 0.08 wt % butylated hydroxytoluene and 0.25 wt % magnesium stearate.

Second, the barrier layer granulations are produced using medium FBG. The composition of barrier-layer granules is comprised of 55 wt % Kollidon, 35 wt % Magnesium Stearate and 10 wt % EMM.

Third, the osmotic push layer granules and barrier layer granules are compressed into a bi-layer tablet with a Multi-layer Korsch press. 350 mg of the osmotic push-layer granules are added and tamped, then 100 mg of barrier layer granules are added onto and finally compressed under a force of 4500 N into a osmotic/barrier bi-layer tablet.

Fourth, 1200 mg of the gabapentin—decyl sulfate complex (500 mg gabapentin equivalent) made according to Example 2, and 500 mg tramadol HCl are dissolved into about 3500 mg propylene glycol (PG) using sonication at 45° C. for 6 h.

Next, Gelatin capsules (size 0) are subcoated with Surelease™. This will inhibit water-permeation into the capsulated liquid formulation during system operation. The subcoating is a membrane of ethylcellulose applied in the form of aqueous dispersion. The dispersion contains 25 wt % solids and is diluted to contain 15 wt % solids by adding purified water. The membrane weight of Surelease™ is 17 mg.

Next, a Surelease™ coated gelatin capsule is separated into two segments (body and cap). The drug-layer composition (520 mg) is filled into the capsule body.

Next, the osmotic/barrier tablet is placed in the filled capsule body. Before inserting the engines into the capsules, a layer of sealing solution is applied around the barrier layer of the gelatin-coated bilayer engines. After engine insertion, a layer of banding solution is applied around the diameter at the interface of capsule and engine. This sealing and banding solution are the same, which is made of water/ethanol 50/50 wt %.

Next, the membrane composition comprising 80% cellulose acetate 398-10 and 20% Pluronic F-68 is dissolved in acetone with solid content of 5% in the coating solution. The solution is sprayed onto the pre-coating assemblies in a 12″ LDCS Hi-coater. After membrane coating, the systems are dried in oven at 45° C. for 24. The assemblies are coated with 131 mg of the rate-controlling membrane.

Next, a 30 mil (0.77 mm) exit orifice is drilled at the drug-layer side using a mechanical drill. By adjusting the membrane weight, the release duration of the systems can be controlled.

### Example 5

Gastric Retention System for Delivery of Gabapentin and Tramadol HCl (150 mg tramadol HCU/350 mg gabapentin)

A dosage form according to the disclosure in U.S. Pat. No. 6,548,083 to Wong et al., granted Apr. 15, 2003, entitled “Prolonged release active agent dosage form adapted for gastric retention”, and incorporated by reference herein in its entirety, is prepared with gabapentin and tramadol HCl.

12.6 grams of gabapentin, 5.4 grams of tramadol HCl, and 3.6 grams of the gel-forming polymer polyethylene oxide, having a number average molecular weight of approximately 8 million grams per mole, are separately screened through a mesh having 40 wires per inch. The polyethylene oxide is supplied under the trade name Polyox.RTM. grade 308 as manufactured by Union Carbide Corporation, Danbury, Conn. The sized active agent and polymer are dry mixed. Then, 8.25 grams of a hydroattractant water-insoluble polymer, hydroxypropyl cellulose having a hydroxypropyl content of 10-13 weight percent and an average fiber particle size of 50 microns, is sieved through the 40-mesh screen and blended into the mixture. The hydroxypropyl cellulose is supplied as Low-Substituted Hydroxypropyl Cellulose grade 11 as manufactured by Shin-Etsu Chemical Company, Ltd., Tokyo, Japan. Anhydrous ethyl alcohol, specially denatured formula 3A, i.e., ethanol denatured with 5 volume percent methanol, is added to the mixture with stirring until a uniformly damp mass formed. This damp mass is extruded with pressure through a screen having 20 wires per inch. The extrudate is then allowed to air dry at room temperature overnight. After drying, the resulting extrudate is passed again through the 20-mesh sieve, forming granules. 0.15 grams of the tableting lubricant, magnesium stearate, are passed through a sieve having 60 wires per inch. The sized 60-mesh lubricant is then tumbled into the granules to produce the finished granulation.

Portions of the resulting granulation are weighed and compacted with caplet-shaped tooling on a Carver press at pressure head of 1.5 tons. Each tablet weighs approximately 833 mg and contains approximately 350 mg gabapentin and 150 mg tramadol HCl. The shape of the tablet has approximately cylindrical proportions. The diameter is approximately 7.6 millimeters (mm) and the length was approximately 22 mm.

A tube of polyolefin material having an outside diameter of 7.7 mm and having a wall thickness of 0.25 mm is sliced with a razor to produce rings. The width of each ring is approximately 3 mm. One ring is then press fitted onto each caplet such that the ring, or band, is located approximately at the midpoint of the length of the caplet.

### Example 6

Matrix Dosage Form for Controlled Delivery of Gabapentin—lauryl sulfate Complex and Tramadol HCl (200 mg tramadol HCl/511 mg gabapentin lauryl sulfate, i.e. 200 mg gabapentin equivalent)

A matrix dosage form according to the present invention is prepared as follows. 143.7 grams of gabapentin—lauryl sulfate complex, prepared as described in Example 2, 56.3 grams of tramadol HCR, 25 grams of hydroxypropyl methylcellulose having a number average molecular weight of 9,200 grams per mole, and 15 grams of hydroxypropyl methylcellulose having a molecular weight of 242,000 grams per mole, are passed through a screen having a mesh size of 40 wires per inch. The celluloses each have an average hydroxyl content of 8 weight percent and an average methoxyl content of 22 weight percent. The resulting sized powders are tumble mixed. Anhydrous ethyl alcohol is added slowly to the mixed powders with stirring until a dough consistency is produced. The damp mass is then extruded through a 20 mesh screen and air dried overnight. The resulting dried material is re-screened through a 20 mesh screen to form the final granules. 2 grams of the tableting lubricant, magnesium stearate, which are sized through an 80 mesh screen, are then tumbled into the granules.

860 mg of the resulting granulation is placed in a die cavity having an inside diameter of 9/32 inch and compressed with deep concave punch tooling using a pressure head of 2 tons. This forms a longitudinal capsule core having an overall length, including the rounded ends, of 0.691 inch. The cylindrical body of the capsule, from tablet land to tablet land, span a distance of 12 mm. Each core contains a unit dose of gabapentin lauryl sulfate complex of 511 mg (200 mg gabapentin equivalent) and 200 mg tramadol HCl.

### Example 7

Modified Matrix Dosage Form for Controlled Delivery of Gabapentin—Lauryl Sulfate Complex and Tramadol HCl (200 mg tramadol HCU511 mg Gabapentin Lauryl Sulfate, i.e. 200 mg Gabapentin Equivalent)

First, the dosage form of Example 6 is provided. Next, rings of polyethylene having an inside diameter of 9/32 inch, a wall thickness of 0.013 inch, and a width of 2 mm are then fabricated. These rings, or bands, are press fitted onto the dosage form of Example 6 to complete the dosage form.

### Example 8

Solid Osmotic Dosage Form for Delivery of a Gabapentin Prodrug and Tramadol HCl (440 mg Gabapentin Prodrug and 150 mg Tramadol HCl)

A dosage form is prepared as follows: Gabapentin acetoxyethyl carbamate is prepared according to Zerangue above.

The gabapentin prodrug and tramadol layer in the dosage form is prepared as follows. First, 6.94 grams of gabapentin prodrug, 2.36 grams of tramadol HCl, 0.50 g polyethylene oxide of 5,000,000 molecular weight, 0.10 g of polyvinylpyrrolidone having molecular weight of about 38,000 are dry blended in a conventional blender for 20 minutes to yield a homogenous blend. Next, denatured anhydrous ethanol is added slowly to the blend with continuous mixing for 5 minutes. The blended wet composition is passed through a 16 mesh screen and dried overnight at room temperature. Then, the dry granules are passed through a 16 mesh screen and 0.10 g magnesium stearate are added and all the dry ingredients are dry blended for 5 minutes. The composition is comprised of 69.4 wt % gabapentin acetoxyethyl carbamate, 23.6 wt % tramadol HCl, 5.0 wt % polyethylene oxide 5,000,000 molecular weight, 1.0 wt % polyvinylpyrrolidone having molecular weight of about 35,000 to 40,000 and 1.0 wt % magnesium stearate.

A push layer comprised of an osmopolymer hydrogel composition is prepared as follows. First, 637.70 g of pharmaceutically acceptable polyethylene oxide comprising a 7,000,000 molecular weight, 300 g sodium chloride and 10 g ferric oxide are separately screened through a 40 mesh screen. The screened ingredients are mixed with 50 g of hydroxypropylmethylcellulose of 9,200 molecular weight to produce a homogenous blend. Next, 150 mL of denatured anhydrous alcohol is added slowly to the blend with continuous mixing for 5 minutes. Then, 0.80 g of butylated hydroxytoluene is added followed by more blending. The freshly prepared granulation is passed through a 20 mesh screen and allowed to dry for 20 hours at room temperature (ambient). The dried ingredients are passed through a 20 mesh screen and 2.50 g of magnesium stearate is added and all the ingredients are blended for 5 minutes. The final composition is comprised of 63.67 wt % of polyethylene oxide, 30.00 wt % sodium chloride, 1.00 wt % ferric oxide, 5.00 wt % hydroxypropylmethylcellulose, 0.08 wt % butylated hydroxytoluene and 0.25 wt % magnesium stearate.

The bi-layer dosage form is prepared as follows. First, 634 mg of the drug layer composition is added to a punch and die set and tamped. Then, 317 mg of the hydrogel composition is added and the two layers compressed under a compression force of 1.0 ton (1000 kg) into a 9/32 inch (0.714 cm) diameter punch die set, forming an intimate bi-layered core (tablet).

A semipermeable wall-forming composition is prepared comprising 80.0 wt % cellulose acetate having a 39.8% acetyl content and 20.0% polyoxyethylene-polyoxypropylene copolymer having a molecular weight of 7680-9510 by dissolving the ingredients in acetone in a 80:20 wt/wt composition to make a 5.0% solids solution. Placing the solution container in a warm water bath during this step accelerates the dissolution of the components. The wall-forming composition is sprayed onto and around the bi-layered core to provide a 60 to 80 mg thickness semi-permeable wall.

Next, a 40 mil (1.02 mm) exit orifice is laser drilled in the semipermeable walled bi-layered tablet to provide contact of the drug containing layer with the exterior of the delivery device. The dosage form is dried to remove any residual solvent and water.

The release rate for the dosage form made according to Example 5 is tested on the Distek 5100 (USP apparatus 2 paddle tester) in 900 mL artificial intestinal fluid (AIF, pH=6.8). The temperature of the dissolution medium was maintained at 37° C. and the paddle speed was 100 rpm. The concentration of gabapentin and of tramadol HCl is measured with HPLC method. Two systems are tested.

What is claimed is:

1. An oral dosage form comprising:
an oral controlled delivery dosing structure comprising structure that controllably delivers tramadol and a substance that comprises gabapentin; and
wherein the weight ratio of gabapentin equivalent to tramadol equivalentpresent in the oral dosage form ranges from about 0.75:1 to about 6.5:1; and
wherein the total weight of the tramadol and substance that comprises gabapentin present in the oral dosage form is less than about 1500 milligrams; and
wherein the oral controlled delivery dosing structure is adapted to controllably deliver the substance that comprises gabapentin at a rate that is effective to, after a single administration of the oral dosage form to a patient, maintain a gabapentin plasma drug concentration that is at least about twenty-five percent of a gabapentin Cmax throughout a window of at least about fifteen hours duration after a time at which the gabapentin Cmax occurs.

2. The oral dosage form of claim 1, wherein the tramadol comprises tramnadol HCl.

3. The oral dosage form of claim 1, wherein the substance that comprises gabapentin comprises a complex that comprises gabapentin and an alkyl sulfate salt.

4. The oral dosage form of claim 1, wherein the window is of at least about eighteen hours duration after the time at which the gabapentin Cmax occurs.

5. The oral dosage form of claim 1, wherein the weight ratio of gabapentin equivalent to tramadol equivalent present in the oral dosage form ranges from about 0.80:1 to about 5.5:1.

6. The oral dosage form of claim 1, wherein the oral dosage form comprises an osmotic oral dosage form.

7. An oral dosage form comprising:
an oral controlled delivery dosing structure comprising structure that controllably delivers tramadol and a substance that comprises gabapentin;
wherein the weight ratio of gabapentin equivalent to tramadol equivalent present in the oral dosage form ranges from about 0.75:1 to about 6.5:1; and
wherein the total weight of the tramadol and substance that comprises gabapentin present in the oral dosage form is less than about 1500 milligrams; and
wherein the controlled delivery dosing structure is adapted to controllably deliver the substance that comprises gabapentin contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs, wherein the wt % is based on the total weight of the substance that comprises gabapentin present in the controlled delivery dosage form.

8. The oral dosage form of claim 7, wherein the tramadol comprises trarnadol HCl.

9. The oral dosage form of claim 7, wherein the substance that comprises gabapentin comprises a complex that comprises gabapentin and an alkyl sulfate salt.

10. The oral dosage form of claim 7, wherein the weight ratio of gabapentin equivalent to tramadol equivalent present in the oral dosage form ranges from about 0.80:1 to about 5.5:1.

11. The oral dosage form of claim 7, wherein the oral dosage form comprises an osmotic oral dosage form.

12. An oral dosage form comprising:
an oral controlled delivery dosing structure comprising structure that controllably delivers tramadol and a substance that comprises gabapentin;
wherein the weight ratio of gabapentin equivalent to tramadol equivalent present in the oral dosage form ranges from about 0.75:1 to about 6.5:1; and
wherein the total weight of the tramadol and substance that comprises gabapentin present in the oral dosage form is less than about 1500 milligrams; and
wherein the controlled delivery dosing structure is adapted to controllably deliver the portion of the substance that comprises tramadol contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs, wherein the wt % is based on the total weight of the tramadol present in the controlled delivery dosage form.

13. The oral dosage form of claim 12, wherein the tramadol comprises tramadol HCl.

14. The oral dosage form of claim 12, wherein the substance that comprises gabapentin comprises a complex that comprises gabapentin and an alkyl sulfate salt.

15. The oral dosage form of claim 12, wherein the weight ratio of gabapentin equivalent to tramadol equivalent present in the oral dosage form ranges from about 0.80:1 to about 5.5:1.

16. The oral dosage form of claim 12, wherein the oral dosage form comprises an osmotic oral dosage form.

17. An oral controlled delivery dosage form comprising
an oral controlled delivery dosing structure comprising structure that controllably delivers:
(i) a substance that comprises gabapentin, and
(ii) tramadol;
wherein the oral controlled delivery dosing structure is adapted to controllably deliver the substance that comprises gabapentin at a release rate that satisfies the following relationship:
Rate₀₋₃=(1/F)*Rate₃₋₁₀
wherein Rate₀₋₃ represents a mean release rate for about a three hour period immediately following administration of the dosage form, Rate₃₋₁₀ represents a mean release rate for a period from about three hours immediately following administration of the oral dosage form to about ten hours immediately following administration of the oral dosage form, and F=X/Y, wherein X=a colonic bioavailability of gabapentin and Y=upper gastrointestinal tract bioavailability of gabapentin.

18. The oral dosage form of claim 17, wherein the tramadol comprises tramadol HCl.

19. The oral dosage form of claim 17, wherein the substance that comprises gabapentin comprises a complex that comprises gabapentin and an alkyl sulfate salt.

20. A method comprising
(1) providing an oral dosage form comprising:
an oral controlled delivery dosing structure comprising structure that controllably delivers tramadol and a substance that comprises gabapentin; and
wherein the weight ratio of gabapentin equivalent to tramadol equivalent present in the oral dosage form ranges from about 0.75:1 to about 6.5:1; and
wherein the total weight of the tramadol and substance that comprises gabapentin present in the oral dosage form is less than about 1500 milligrams; and
wherein the oral controlled delivery dosing structure is adapted to controllably deliver the substance that comprises gabapentin at a rate that is effective to, after a single administration of the oral dosage form to a patient, maintain a gabapentin plasma-drug concentration that is at least about twenty five percent of a gabapentin Cmax throughout a window of at least about fifteen hours duration after a time at which the gabapentin Cmax occurs; and
(2) administering the oral dosage form to a patient.

21. The method of claim 20, wherein the tramadol comprises tramadol HCl.

22. The method of claim 20, wherein the substance that comprises gabapentin comprises a complex that comprises gabapentin and an alkyl sulfate salt.

23. The method of claim 20, wherein the window is of at least about eighteen hours duration after the time at which the gabapentin Cmax occurs

24. The method of claim 20, wherein the weight ratio of gabapentin equivalent to tramadol equivalent present in the oral dosage form ranges from about 0.80:1 to about 5.5:1.

25. The method of claim 20, wherein the oral dosage form comprises an osmotic oral dosage form.

26. A method comprising:
(1) providing an oral dosage form comprising:
an oral controlled delivery dosing structure comprising structure that controllably delivers tramadol and a substance that comprises gabapentin;
wherein the weight ratio of gabapentin equivalent to tramadol equivalent present in the oral dosage form ranges from about 0.75:1 to about 6.5:1; and
wherein the total weight of the tramadol and substance that comprises gabapentin present in the oral dosage form is less than about 1500 milligrams; and
wherein the controlled delivery dosing structure is adapted to controllably deliver the substance that comprises gabapentin contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs, wherein the wt % is based on the total weight of the substance that comprises gabapentin present in the controlled delivery dosage form; and
(2) administering the oral dosage form to a patient.

27. The method of claim 26, wherein the tramadol comprises tramadol HCl.

28. The method of claim 26, wherein the substance that comprises gabapentin comprises a complex that comprises gabapentin and an alkyl sulfate salt.

29. The method of claim 26, wherein the weight ratio of gabapentin equivalent to tramadol equivalent present in the oral dosage form ranges from about 0.80:1 to about 5.5:1.

30. The method of claim 26, wherein the oral dosage form comprises an osmotic oral dosage form.

31. A method comprising:
(1) providing an oral dosage form comprising:
an oral controlled delivery dosing structure comprising structure that controllably delivers tramadol and a substance that comprises gabapentin;
wherein the weight ratio of gabapentin equivalent to tramadol equivalent present in the oral dosage form ranges from about 0.75:1 to about 6.5:1; and
wherein the total weight of the tramadol and substance that comprises gabapentin present in the oral dosage form is less than about 1500 milligrams; and
wherein the controlled delivery dosing structure is adapted to controllably deliver the portion of the substance that comprises tramadol contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs, wherein the wt % is based on the total weight of the tramadol present in the controlled delivery dosage form; and
(2) administering the oral dosage form to a patient.

32. The method of claim 31, wherein the tramadol comprises tramadol HCl.

33. The method of claim 31, wherein the substance that comprises gabapentin comprises a complex that comprises gabapentin and an alkyl sulfate salt.

34. The method of claim 31, wherein the weight ratio of gabapentin equivalent to tramadol equivalent present in the oral dosage form ranges from about 0.80:1 to about 5.5:1.

35. The method of claim 31, wherein the oral dosage form comprises an osmotic oral dosage form.

36. A method comprising:
(1) providing an oral controlled delivery dosage form comprising:
an oral controlled delivery dosing structure comprising structure that controllably delivers:
(i) a substance that comprises gabapentin, and
(ii) tramadol;
wherein the oral controlled delivery dosing structure is adapted to controllably deliver the substance that comprises gabapentin at a release rate that satisfies the following relationship:
Rate₀₋₃=(1/F)*Rate₃₋₁₀
wherein Rate₀₋₃ represents a mean release rate for about a three hour period immediately following administration of the dosage form, Rate₃₋₁₀ represents a mean release rate for a period from about three hours immediately following administration of the oral dosage form to about ten hours immediately following administration of the oral dosage form, and F=X/Y, wherein X=a colonic bioavailability of gabapentin and Y=upper gastrointestinal tract bioavailability of gabapentin; and
(2) administering the dosage form to a patient.

37. The method of claim 36, wherein the tramadol comprises tramadol HCl.

38. The method of claim 36, wherein the substance that comprises gabapentin comprises a complex that comprises gabapentin and an alkyl sulfate salt.

39. A pharmaceutical composition comprising
a substance comprising a complex that comprises (i) gabapentin and (ii) a transport moiety; and
tramadol.

40. The pharmaceutical composition of claim 39, wherein the transport moiety comprises an alkyl sulfate salt.

41. The pharmaceutical composition of claim 40, wherein the alkyl sulfate salt comprises sodium lauryl sulfate.

42. The pharmaceutical composition of claim 39, with the proviso that the substance excludes substances that comprise gabapentin prodrugs wherein the gabapentin prodrug comprises chemical structure that enhances colonic absorption of the gabapentin prodrug as compared to gabapentin.

43. An oral dosage form comprising the pharmaceutical composition of claim 39.

44. The oral dosage form of claim 43, wherein the oral dosage form comprises an oral controlled delivery dosage form.

45. The oral dosage form of claim 44, wherein the oral dosage form comprises an osmotic oral controlled delivery dosage form.

46. The oral dosage form of claim 45, wherein the osmotic oral controlled delivery dosage form comprises a solid osmotic oral controlled delivery dosage form.

47. The oral dosage form of claim 45, wherein the osmotic oral controlled delivery dosage form comprises a liquid osmotic oral controlled delivery dosage form.

48. A method comprising:
(1) providing a pharmaceutical composition comprising a substance comprising a complex that comprises (i) gabapentin and (ii) a transport moiety; and tramadol; and
(2) administering the pharmaceutical composition to a patient.

49. The method of claim 48, wherein the transport moiety comprises an alkyl sulfate salt.

50. The method of claim 48, wherein the alkyl sulfate salt comprises sodium lauryl sulfate.

51. The method of claim 48, with the proviso that the substance excludes substances that comprise gabapentin prodrugs wherein the gabapentin prodrug comprises chemical structure that enhances colonic absorption of the gabapentin prodrug as compared to gabapentin.

52. A method comprising
(1) providing the oral dosage form of claim 43; and
(2) administering the oral dosage form to a patient.

53. The method of claim 52, wherein the oral dosage form comprises an oral controlled delivery dosage form.

54. The method of claim 53, wherein the oral dosage form comprises an osmotic oral controlled delivery dosage form.

55. The method of claim 54, wherein the osmotic oral controlled delivery dosage form comprises a solid osmotic oral controlled delivery dosage form.

56. The method of claim 54, wherein the osmotic oral controlled delivery dosage form comprises a liquid osmotic oral controlled delivery dosage form.
